(19) United States
(12) Patent Application Publication
(10) Pub. No.: US 2026/0055403 A1
(43) Pub. Date: Feb. 26, 2026

(54) TREATMENT OF ALZHEIMER'S DISEASE WITH VASCULAR PROGENITOR-DERIVED EXOSOMES ENRICHED WITH MICRORNAS OR THIOREDOXIN

(71) Applicant: University of Maryland, Baltimore, Baltimore, MD (US)

(72) Inventors: Peixin YANG, Ellicott City, MD (US); Wei-Bin SHEN, Ellicott City, MD (US)

(73) Assignee: University of Maryland, Baltimore, Baltimore, MD (US)

(21) Appl. No.: 19/103,359

(22) PCT Filed: Aug. 17, 2023
(86) PCT No.: PCT/US2023/030426

§ 371 Date: Feb. 12, 2025

Related U.S. Application Data
(60) Provisional application No. 63/399,348, filed on Aug. 19, 2022.

(51) Int. Cl. C12N 15/113; A61P 25/28

(52) U.S. Cl. CPC C12N 15/113; A61P 25/28; C12N 2310/141

(57) ABSTRACT

Methods for treating and/or preventing Alzheimer's disease via administration of thioredoxin-1 (Trx1) mRNA- or miR-551b miR-NA-containing exosomes derived from Flk-1+ vascular endothelial progenitor cells are reported. Further, wherein the subject carries the APOE E4 risk factor gene; wherein the subject also has mild cognitive impairment (MCI); and wherein the Flk-1+ exosomes are derived from vascular endothelial progenitor cells or the exosomes are derived from neural stem cells.

### BACKGROUND OF INVENTION

Alzheimer's disease (AD) affects over 5.7 million Americans. The seriousness of this disease is further manifested by the fact that 1 in 10 people who are >65 years older has AD, and this AD prevalence will triple by 2050 [9]. Despite great progress in understanding the pathogenesis of AD, there is still no disease-modifying treatment available [10, 11]. Studies in human familial AD have revealed that mutations in the presenilin proteins (PSEN1; PSEN2) and amyloid precursor protein (APP) contribute to the accumulation of amyloid-beta (Aβ) protein [12, 13], which is a hallmark of AD progression that leads to eventual cognitive impairment. As the majority of AD cases are not hereditary, environmental and genetic factors may act together to lead to AD development.

AD and insulin resistance are two related aging diseases [14]. Brain glucose hypometabolism is observed decades prior to AD manifestation [15-17]. While a causal relationship between systemic insulin resistance and AD has not been established, brain insulin resistance in the absence of type 2 diabetes has been proposed as a contributor to AD [18]. AD brains exhibit lower insulin receptor (IR) expression and impaired IR signaling compared to those of age-matched controls [19, 20]. Furthermore, an ex vivo study using postmortem brain tissues convincingly demonstrated insulin resistance in AD tissues without a history of diabetes [21]. An increase in brain insulin levels has been shown to improve cognitive function in AD [22, 23]. However, the cause of insulin resistance in AD is unknown, and whether insulin resistance is a causal factor of AD has not yet been established.

Oxidative stress precedes Aβ deposition [24, 25], and oxidative stress-induced kinase signaling causes insulin resistance [26]. Previous studies have revealed that diabetes-induced oxidative stress activates apoptosis signal-regulating kinase 1 (ASK1) by oxidizing the ASK1 endogenous inhibitor thioredoxin 1 (Trx1) in early neural development [27]. A recent study showed that ASK1 deficiency improved cognitive function and reduced soluble Aβ levels in an AD mouse model [1]. Thus, it is possible that early ASK1 activation may contribute to brain insulin resistance in AD.

Novel methods for increasing Trx1 activity and/or decreasing ASK1 activity in the brains of subjects having AD, or at risk for developing AD, might provide therapeutic benefits to such subjects. The present invention is directed to these and other important goals.

### BRIEF SUMMARY OF INVENTION

The present inventors have found that decreased Trx1 expression and activation of the ASK1-p38MAPK pathway were present in postmortem Alzheimer's disease (AD) frontal cortices and hippocampi with the co-occurrence of IR signaling impairment. Likewise, the frontal cortices and hippocampi from non-AD individuals who were carriers of APOE ε4 variants, the strongest genetic risk factor for AD [28], exhibited Trx1 reduction and ASK1 activation with reduced IR signaling compared to those of noncarrier controls. In AD mouse models with transgenic expression of human APP and PSEN1 mutations, the inventors found that Trx1 reduction and ASK1 activation preceded IR signaling impairment, which occurred earlier than Aβ senile plaques and cognitive dysfunction. The same sequence of events, during neuronal differentiation from induced pluripotent stem cells (iPSCs) of AD patients, in which ASK1 was activated earlier than IR signaling impairment, which preceded Aβ deposition, was observed. Thus, the inventors hypothesize that activation of the ASK1-p38MAPK pathway due to Trx1 reduction causes brain insulin resistance through downregulation of a microRNA, miR-551b, and that these cellular events participate in AD pathogenesis and progression. Ask1 gene deletion, as well as overexpression of Trx1 or miR-551b via exosomes and transgenic mice prevent brain insulin resistance leading to AD syndrome amelioration.

The present invention is based on these discoveries and generally encompasses therapeutic applications for Alzheimer's disease AD-related dementia.

More specifically, and in a first embodiment, the present invention is directed to methods of treating and/or preventing Alzheimer's disease and AD-related dementia. The methods comprise administering a therapeutically-effective amount of exosomes to a subject in need thereof, wherein the exosomes contain a therapeutic agent for Alzheimer's disease and/or AD-related dementia.

In a related embodiment, the invention is directed to methods of delaying onset of symptoms of Alzheimer's disease and AD-related dementia. The methods comprise administering a therapeutically-effective amount of exosomes to a subject in need thereof, wherein the exosomes contain a therapeutic agent for Alzheimer's disease and/or AD-related dementia.

In certain aspects of these embodiments, the therapeutic agent is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitors including selonsertib, p38MARK CRISPR/Cas9 and gRNA, p38MAPK siRNA, and p38MAPK inhibitors including Ralimetinib. In particular aspects, the therapeutic agent is Trx1 mRNA, Trx1 protein, Trx1 core peptide, miR-551b, miR-383, or miR-873.

In certain aspects of these embodiments, the subject carries the APOE ε4 risk factor gene. In certain other aspects of these embodiments, the subject also has mild cognitive impairment (MCI).

In certain aspects of these embodiments, the exosomes are derived from Flk-1⁻ progenitor cells. In particular, non-limiting aspects, the Flk-1⁺ exosomes are derived from vascular endothelial progenitor cells or the exosomes are derived from neural stem cells.

In certain aspects of these embodiments, the exosomes are administered to the subject via an intravenous injection or other avenues such as skin patches.

In a second embodiment, the invention is directed to methods of suppressing ASK1 activity in the brain of a subject. The methods comprise administering a therapeutically-effective amount of exosomes to a subject in need thereof, wherein the exosomes contain a therapeutic agent that inhibits ASK1 gene transcription, inhibits ASK1 mRNA translation, or inhibits ASK1 protein activity.

In certain aspects of this embodiment, the therapeutic agent is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitors including selonsertib, p38MARK CRISPR/Cas9 and gRNA, p38MAPK siRNA, and p38MAPK inhibitors including Ralimetinib. In particular aspects, the therapeutic agent is Trx1 mRNA, Trx1 protein, Trx1 core peptide, miR-551b, miR-383, or miR-873.

In certain aspects of this embodiment, the subject has Alzheimer's disease (AD), mild cognitive impairment (MCI), or another form of AD-related dementia, or the subject is at risk of developing Alzheimer's disease. In particular aspects, the subject carries the APOE ε4 risk factor gene. In certain other aspects, the subject also has mild cognitive impairment (MCI)

In certain aspects of this embodiment, the exosomes are derived from Flk-1⁺ progenitor cells. In particular, non-limiting aspects, the Flk-1⁺ exosomes are derived from vascular endothelial progenitor cells or the exosomes are derived from neural stem cells.

In certain aspects of this embodiment, the exosomes are administered to the subject via an intravenous injection and other avenues such as skin patches.

In a third embodiment, the invention is directed to methods of inducing glucose metabolism in a neuron or neuronal cell. The methods comprise contacting a neuron or neuronal cell with an exosome, wherein the exosomes contain an agent having glucose metabolism inducing activity.

In a related embodiment, the invention is directed to methods of inducing glucose metabolism in a neuron or neuronal cell in a subject. These methods comprise administering a therapeutically-effective amount of exosomes to a subject in need thereof, wherein the exosomes contain an agent having glucose metabolism inducing activity.

In certain aspects of these embodiments, the agent is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitors including selonsertib, p38MARK CRISPR/Cas9 and gRNA, p38MAPK siRNA, and p38MAPK inhibitors including Ralimetinib. In particular aspects, the therapeutic agent is Trx1 mRNA, Trx1 protein, Trx1 core peptide, miR-551b, miR-383, or miR-873.

In certain aspects of these embodiments, the subject has Alzheimer's disease (AD), mild cognitive impairment (MCI), or another form of AD-related dementia, or the subject is at risk of developing Alzheimer's disease. In particular aspects, the subject carries the APOE ε4 risk factor gene. In certain other aspects, the subject also has mild cognitive impairment (MCI)

In certain aspects of these embodiments, the exosomes are derived from Flk-1⁻ progenitor cells. In particular, non-limiting aspects, the Flk-1⁺ exosomes are derived from vascular endothelial progenitor cells or the exosomes are derived from neural stem cells.

In certain aspects of these embodiments, the exosomes are administered to the subject via an intravenous injection or other avenues such as skin patches.

In a fourth embodiment, the invention is generally directed to methods for delivering a biological molecule to a neuron or neuronal cell of a subject. The methods comprise administering exosomes to the subject, wherein the exosomes contain a biological molecule. The neuron or neuronal cell may be in the brain of the subject.

In certain aspects of this embodiment, the biological molecule is one or more of a nucleotide, a oligonucleotide, polynucleotide, DNA, RNA, mRNA, microRNA, siRNA, cDNA, an amino acid, a peptide, a polypeptide, a protein, a lipid, a fatty acid, a glycolipid, a sterol, a monosaccharide, an oligosaccharide, a polysaccharide, a vitamin, small molecule inhibitors, a FDA approved drug, a growth factor, and anti-apoptotic factor, or a hormone.

In certain aspects of this embodiment, the exosomes are derived from Flk-1⁺ progenitor cells. In particular, non-limiting aspects, the Flk-1⁺ exosomes are derived from vascular endothelial progenitor cells or the exosomes are derived from neural stem cells.

In certain aspects of this embodiment, the exosomes are administered to the subject via an intravenous injection or other avenues such as skin patches.

In certain aspects of this embodiment, the delivery is targeted delivery, for example in which the exosomes target neurons or neuronal cells in the brain.

In relevant aspects and embodiments of the invention, the therapeutically-effective amount of exosomes is a population of between 1×10⁶ and 1×10¹² exosomes, administered daily or once a week. In certain aspects, the therapeutically-effective amount of exosomes is a population of 5×10⁹ exosomes.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Illustration of the central hypothesis. Before overt AD neuropathology occurs (pre-AD), elevated oxidative stress suppresses Trx1 and other antioxidant expression leading to the dissociation of Trx1 from ASK1. This event leads to the activation of the ASK1-p38MAPK pathway, which represses the expression of miR-551b and other miRNAs leading to brain insulin resistance, which results in AD manifestation. Dashed light blue arrows indicate alternative routes.

FIG. 2. High-fat diet (HFD) induces systemic insulin resistance and accelerates AD neuropathology. Control non-AD (Ctrl) and APP/PS1 pre-AD mice were fed an HFD (60% fat, Research Diets) or a normal diet (10% fat) from 4 weeks old to 3.5 months old when glucose tolerance tests (A) and Morris Water Maze (MWM) for the assessment of spatial learning (B,C). B is the Acquisition Learning. After 3 day training, pre-AD+HFD mice display impaired learning illustrated by significantly greater time to find the platform. C is the Probe Trial. Pre-AD+HFD mice have impaired recall in the MWM indicated by reduced time in the target quadrant when tested without the platform (n=5). * indicate significant difference compared to the other groups in one-way ANOVA followed by a Tukey's test (P<0.05).

FIG. 3. ASK1 activation (phosphorylation, p-ASK1) (A) and reduced Akt activity (p-Akt) (B) in postmortem frontal cortices of AD patients. p-Akt is the major readout of the insulin signaling. Immunofluorescent staining was performed on four cases of control non-AD (ctrl) and four cases of AD cases (n=4). Intensity of the fluorescent signal was quantified by the NIH Image J. The staining signal (green) was normalized by DAPI. * indicate significant difference compared to the control group in Student's t test (P<0.05). Data in hippocampi were not included.

FIG. 4. ASK1 activation (phosphorylation, p-ASK1) (A) and impaired insulin signaling in postmortem frontal cortices from non-AD individuals with APOE ε4 variants or APOE ε3 (control, ctrl). Immunofluorescent staining for the phosphorylation of ASK1, IRβ (B), IRS-1 at a serine residue (Ser-IRS-1) (C) and Akt (D) was performed in four cases of control and four cases of non-AD APOE ε4 individuals (n=4). p-Ser-IRS-1 is an inhibitory marker for insulin signaling. Intensity of the fluorescent signal was quantified by the NIH Image J. The staining signal (green) was normalized by DAPI (%). * indicate significant difference compared to the control group in Student's t test (P<0.05). Data in hippocampi were not included.

FIG. 5. ASK1 activation and impaired insulin signaling precede overt AD neuropathology in APP/PS1 AD mice. (A) Aβ deposits were observed only in the frontal cortices of APP/PS1 AD mice (n=3) at 5.5 months but not at 3.5 months of age. Immunofluorescent staining signals for phosphorylation of ASK1 (B) and IRS-1 at a serine residue (Ser-IRS-1) (C), an inhibitory marker of insulin signaling, were observed in AD mice at 3.5 months of age. Intensity of the fluorescent signal was quantified by the NIH Image J. The staining signal (red) was normalized by DAPI. * indicate significant difference compared to the control non-AD group (ctrl) in Student's t test (P<0.05). Data in hippocampi were not included.

FIG. 6. ASK1 activation and impaired insulin signaling in AD iPSCs. Immunofluorescence staining for phosphorylation of ASK1 (A), IRβ (B) and Akt (C) was performed in control non-AD (ctrl) and AD human iPSCs (n=6). Intensity of the staining signal (green) was quantified by the NIH Image J. DAPI (4′,6-diamidino-2-phenylindole) staining for cell nuclei (blue). * indicate significant difference compared to the control group in Student's t test (P<0.05).

FIG. 7. p38MAPK activation (phosphorylation, p-p38MAPK) in frontal cortices of non-AD individuals with APOE ε4 variants (A) and 3.5-month-old APP/PS1 AD mice (B). Intensity of the staining signal (green) for p-p38MAPK (A) (n=4 cases/group) and immunoblotting bands for p-p38MAPK in both female (n=3 mice/group) and male (n=3 mice/group, data not shown) (B) was quantified by the NIH Image J (A,B) and normalized by DAPI (%, A). * indicate significant difference compared to the control (ctrl) group (APOE ε3 in A and control non-AD mice in B) in Student's t test (P<0.05).

FIG. 8. The oxidative stress marker 4-HNE is elevated in postmortem frontal cortices from non-AD individuals with APOE ε4 variants (A) and from AD patients (B). Immunofluorescence staining for 4-HNE was conducted in four cases/group (n=4). Intensity of the staining signal (red) was quantified by the NIH Image J. * indicate significant difference compared to the control groups (APOE 3 in A and non-AD control in B) in Student's t test (P<0.05).

FIG. 9. Thioredoxin 1 (Trx1) is decreased in postmortem frontal cortices from non-AD individuals with APOE ε4 variants (A), AD patients (B) and APP/PS1 AD mice (C). Immunofluorescence staining for Trx1 was conducted in four cases/group (n=4) in A and B. Intensity of the staining signal (green) and immunoblotting bands (C) was quantified by the NIH Image J. In D, Trx1 mRNA levels. * indicate significant difference compared to the control (ctrl) groups (APOE 3 in A, non-AD control in B and non-AD control mice in C and D (n=3 mice/group)) in Student's t test (P<0.05). Data in hippocampi were not included.

FIG. 10. Flk1+ progenitor exosomes delivered to mice via tail-vein injections reach the brain and are taken up by cells. 109 green fluorescence-labeled exosomes were injected to 3-month-old mice (n=3 mice) and after 24 hours, brain sections were examined for the presence of green-labeled exosomes. Exosomes were present in all three injected mice and representative imaging is shown.

FIG. 11. Tail-vein injections of Trx1 mRNA-loaded exosomes increase Trx1 expression and suppress the activation of the ASK1-p38MAPK pathway. 109 exosomes per day for two days with Trx1 mRNA loading (EXO+Trx1) or without control mRNA loading (EXO+ctrl) were injected to 3-month-old APP/PS1 AD mice (n=4 mice), and 24 hours after the last injections, brain sections were examined for the presence of green-labeled exosomes and Trx1 protein expression in A and flag-tagged protein due to Flag-tagged Trx1 mRNA in B. Intensity of the fluorescent signal in A and B was quantified by the NIH Image J and normalized by DAPI (blue). Quantification data were plotted in the bar graphs of A and B (n=4 mice/group). In C, immunoblotting of Trx1, p-ASK1, p-p38MAKP and p-GSK-1β with density quantification showing on the top of the bands. p-GSK-1β is the inactive form, a readout of high insulin signaling.

FIG. 12. An miRNA profiling study reveals the downregulation of a group of miRNAs in postmortem tissue samples of frontal cortices from non-AD individuals with APOE ε4 variants (E4) compared with those from individuals with APOE 3 (control, ctrl). In A, a heatmap shows the differential miRNA expression in these two groups (n=4 in the E4 group and n=3 in the ctrl group). In B, the top downregulated miRNAs with mouse homologs. In C, three miRNAs that are reported to positively regulate insulin signaling.

FIG. 13. A transcriptome microarray study reveals the top cellular pathways affected in postmortem tissue samples of frontal cortices from non-AD individuals with APOE ε4 variants (E4) compared with those from individuals with APOE ε3 (control, ctrl). Four samples each group (n=4) were used in human transcriptome microarray chips.

FIG. 14. Tail-vein delivery of miR-551b-loaded exosomes (Exo) improves performance in the Morris Water Maze test in AD mice. Male AD mice at 5.0 months of age were tail-vein injected daily for five days with 109 exosomes (Exo, control) or exosomes transfected with miR-551b mimics (Exo+miR551b). AD mice injected with the control exosomes (Green line) displayed decreased performance compared with non-AD mice (black line) during the training period of the Morris Water Maze test. Injection of Exo+miR551b, one dose a day for 5 days, to replenishing miR-551b-3p rescue the learning capability. Four mice were used in each group (n=6). * indicate significant difference compared to the other two groups in Student's t-test (P<0.05).

FIG. 15. miR-551b is downregulated in AD mice before overt AD pathology (A) and in human AD iPSCs. Trx1 overexpression (B) and the ASK1 inhibitor selonsertib (B) restore miR-551b expression in human AD iPSCs. In A, miR-551b was downregulated in the hippocampi of 1-, 1.5- and 3.5-month-old APP/PS1 AD mice (n=4 mice/group/time points). In B and C, miR-551b levels in non-AD control (ctrl) and AD human iPSCs. * indicate significant difference compared to the control (ctrl) non-AD mouse group (A) in Student's t test (P<0.05). In B and C, * indicate significant difference compared to the other groups in one-way ANOVA followed by a Tukey's test (P<0.05).

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention is generally directed to therapeutic techniques for treating and/or preventing Alzheimer's disease (AD), mild cognitive impairment (MCI), or another form of AD-related dementia, or a subject at risk of developing Alzheimer's disease via administration of exosomes derived from Flk-1⁺ vascular endothelial progenitor cells or neural stem cells that contain therapeutic agents, such as thioredoxin-1 (Trx1) mRNA or miR-551b.

As discussed in detail below, accumulating experimental evidence supports the crucial role of oxidative stress in AD pathogenesis [32-36]. Reactive oxygen species (ROS) activate the kinase ASK1 by oxidizing its endogenous inhibitor, Trx1 [37]. Prior research has demonstrated that the oxidative stress responsive ASK1 impairs insulin signaling in cultured cells [38]. Furthermore, the ASK1 downstream kinase, p38MAPK, also inhibits insulin signaling [39]. Based on these prior findings and the preliminary data presented herein, the inventors hypothesize the ASK1-p38MAPK pathway is responsible for insulin signaling impairment during AD pathogenesis.

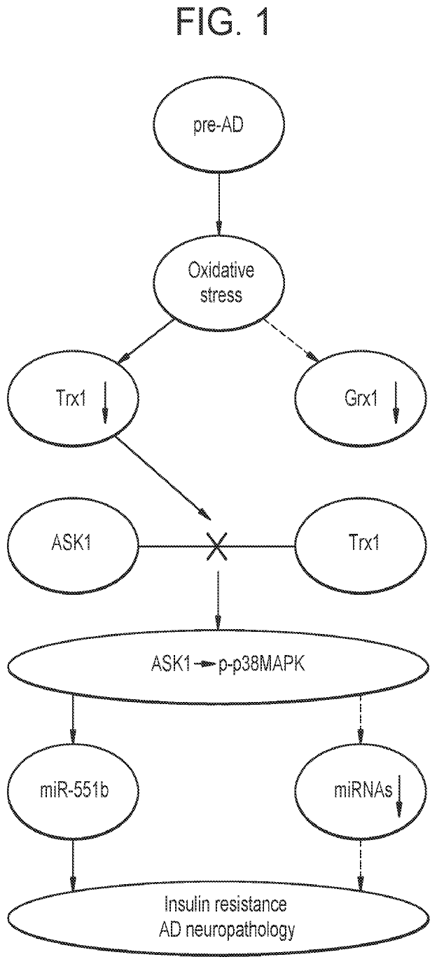

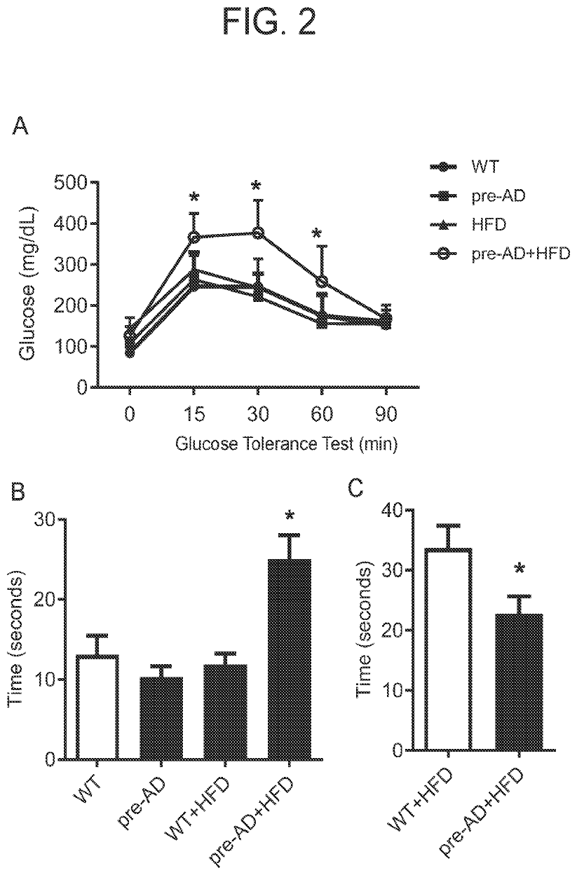

Reduced insulin receptor expression and binding have been observed in the brains of AD patients [19, 20] and are associated with AD severity [19]. Numerous studies have consistently shown that impaired insulin signaling is present in postmortem AD brain tissues [19, 21, 40-47]. An elegant ex vivo insulin-signaling-stimulation study using post-mortem hippocampal tissues directly demonstrated insulin resistance in AD tissues [21]. Thus, brain insulin resistance accounts for glucose hypometabolism in AD, as proposed 25 years ago [18]. However, the molecular mechanisms underlying insulin resistance in AD and a possible causal relationship between insulin resistance and AD pathology have not yet been determined. The inventors hypothesize that the ASK1-p38MAPK pathway inhibits insulin receptor signaling by suppressing the miRNA miR-551b (FIG. 1). The preliminary data demonstrate that the feeding of a high-fat diet (HFD), a regime to induce systemic insulin resistance including brain insulin resistance [4, 5], accelerated overt AD onset in an AD mouse model (FIG. 2), supporting a role of brain insulin resistance in AD pathogenesis. Although the role of hyperglycemia in the effect of HFD feeding cannot be totally ruled out, brain insulin resistance is induced very early under HFD feeding in mice [4].

Drugs targeting the process of amyloidogenesis, as proposed in the amyloid hypothesis, do not exert significant efficacy in clinical trials [49-53]. Among FDA-approved drugs, three acetylcholinesterase inhibitors and one N-methyl-D-aspartate receptor antagonist are palliative medications with only temporary alleviation of cognitive symptoms in AD [49]. Thus, treatments that aim to tackle the underlying cause of AD are lacking. The investigation of pathology other than amyloidogenesis may reveal compelling new drug targets. The experiments discussed herein will provide mechanistic insights for the development of inhibitors of ASK1 and p38MAPK, insulin, insulin-sensitizing medicines, Trx1 and miR-551b via specific exosome delivery. Indeed, intranasal insulin administration improves memory functions in AD patients [22, 23, 54]. Additionally, insulin sensitizers, namely, glucagon-like peptide 1-targeting drugs, show promise in early AD clinical trial studies [55]. The newly developed ASK1 inhibitor selonsertib reduces liver fibrosis in patients with nonalcoholic steatohepatitis [56], could tested in AD models if ASK1 is critical involved in AD pathogenesis. It has been shown that p38MAPK activity is increased in AD brains [57]. The inhibition of p38MAPK via specific inhibitors has emerged as a potential therapy for AD. Recent pilot studies have shown that a p38MAPK inhibitor improves episodic memory function and reverses synaptic dysfunction in AD patients [58, 59].

Exosomes, which are small extracellular vesicles ranging from 50 to 200 nm in diameter, enable intercellular communication through their exosomal cargos. Released neuronal exosomes in AD patients contain a large amount of Aβ and phosphorylated tau [60, 61], suggesting that cell-autonomous protective processes occur in neurons during AD-associated cytotoxicity. The delivery of non-AD neuronal exosomes into the brain of APP transgenic mice suppresses Aβ generation and deposition [62], underscoring the role of neuronal exosomes in Aβ clearance [62] and suggesting that reduced neuronal exosome release may contribute to the development of AD pathology. It has been shown that exosomes can cross the blood-brain barrier and promote functional recovery in animal models of stroke [63, 64]. Indeed, improving Aβ clearance by exosome administration may provide a novel therapeutic intervention for AD [65]. As discussed herein, the present inventors have found that exosomes from Flk1-positive (Flk1+) vascular progenitors specifically targeted cells in the brain (FIG. 10, 11). Trx1 mRNA- or miR-551b-enriched exosomes are used to examine whether the suppression of ASK1 via Trx1 or the restoration of miR-551b expression rescues insulin signaling/sensitivity and thus ameliorates AD pathology. Therefore, the present invention covers the spectrum from mechanistic studies to therapeutic interventions to further elucidate the etiology of AD.

The present invention explores a number of new concepts. First, the concept that the ASK1-p38MAPK pathway is responsible for impaired insulin signaling preceding overt AD pathology is a novel perspective. Second, the hypothesis that inhibiting ASK1 activation via exosomal delivery of the mRNA of the endogenous ASK1 inhibitor Trx1 to restore insulin signaling and potentially lead to the alleviation of AD pathology is an innovative approach. Third, the hypothesis that the activation of the ASK1-p38MAPK pathway leads to the downregulation of miR-551b is a novel idea. Fourth, it is a novel hypothesis that miR-551b reduction results in insulin signaling impairment, brain insulin resistance and AD pathology. By extension, testing the ability of exosomal delivery of miR-551b to restore insulin signaling and alleviate AD pathogenesis is an original strategy.

In light of these findings, the present invention was realized. As suggested above, the invention is directed to methods based on the delivery of therapeutically-effective amounts of exosomes containing therapeutic agents to the brains of subjects having Alzheimer's disease, AD-related dementia or at risk of developing Alzheimer's disease. The therapeutic agents generally increase Trx proteins levels or activity of the protein, or decrease ASK1 protein levels or activity of the protein, in the brain of the subjects.

In a first embodiment, the invention is directed to methods of treating and/or preventing Alzheimer's disease and AD-related dementia. The methods comprise administering a therapeutically-effective amount of exosomes to a subject in need thereof, wherein the exosomes contain a therapeutic agent for Alzheimer's disease and/or AD-related dementia.

In a related embodiment, the invention is directed to methods of delaying onset of symptoms of Alzheimer's disease and AD-related dementia. The methods comprise administering a therapeutically-effective amount of exosomes to a subject in need thereof, wherein the exosomes contain a therapeutic agent for Alzheimer's disease and/or AD-related dementia.

In a second embodiment, the invention is directed to methods of suppressing ASK1 activity in the brain of a subject. The methods comprise administering a therapeutically-effective amount of exosomes to a subject in need thereof, wherein the exosomes contain a therapeutic agent that inhibits ASK1 gene transcription, inhibits ASK1 mRNA translation, or inhibits ASK1 protein activity.

In a third embodiment, the invention is directed to methods of inducing glucose metabolism in a neuron or neuronal cell. The methods comprise contacting a neuron or neuronal cell with an exosome, wherein the exosomes contain an agent having glucose metabolism inducing activity.

In a related embodiment, the invention is directed to methods of inducing glucose metabolism in a neuron or neuronal cell in a subject. These methods comprise administering a therapeutically-effective amount of exosomes to a subject in need thereof, wherein the exosomes contain an agent having glucose metabolism inducing activity.

In a fourth embodiment, the invention is generally directed to methods for delivering a biological molecule to a neuron or neuronal cell of a subject. The methods comprise administering exosomes to the subject, wherein the exosomes contain a biological molecule. The neuron or neuronal cell may be in the brain of the subject.

In each of the relevant aspects and embodiments of the invention, the therapeutic agents for treatment and/or prevention of Alzheimer's disease is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitors including selonsertib, p38MARK CRISPR/Cas9 and gRNA, p38MAPK siRNA, and p38MAPK inhibitors including Ralimetinib. In particular aspects, the therapeutic agent is Trx1 mRNA, Trx1 protein, Trx1 core peptide, miR-551b, miR-383, or miR-873.

In each of the relevant aspects and embodiments of the invention, the therapeutic agents for inhibiting ASK1 gene transcription, inhibiting ASK1 mRNA translation, or inhibiting ASK1 protein activity is one or more of thioredoxin-1 (Trx1) mRNA, Trx1 protein, Trx1 core peptide, miR-551b miR-383, miR-873, apoptosis signal-regulating kinase 1 (ASK1) CRISPR/Cas9 and gRNA, ASK1 siRNA, ASK1 inhibitor, p38 MAPK CRISPR/Cas9 and gRNA, p38MARK siRNA, and p38MAPK inhibitor. In particular aspects, the therapeutic agent is Trx1 mRNA, Trx1 protein, or Trx1 core peptide.

In each of the relevant aspects and embodiments of the invention, the agent having glucose metabolism inducing activity is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitor, p38MAPK CRISPR/Cas9 and gRNA, p38MARK siRNA, and p38MAPK inhibitor. In particular aspects, the agent is Trx1 mRNA, Trx1 protein, or Trx1 core peptide.

In each of the relevant aspects and embodiments of the invention, the biological molecule that may be contained within an exosome for delivery to a neuron or neuronal cell is one or more of a nucleotide, a oligonucleotide, polynucleotide, DNA, RNA, mRNA, microRNA, siRNA, cDNA, an amino acid, a peptide, a polypeptide, a protein, a lipid, a fatty acid, a glycolipid, a sterol, a monosaccharide, an oligosaccharide, a polysaccharide, a vitamin, small molecule inhibitors, a FDA approved drug, a growth factor, and anti-apoptotic factor, or a hormone.

In each of the relevant aspects and embodiments of the invention, the subject has Alzheimer's disease (AD), mild cognitive impairment (MCI), or another form of AD-related dementia, or the subject is at risk of developing Alzheimer's disease. In particular aspects, the subject carries the APOE ε4 risk factor gene. In certain other aspects, the subject also has mild cognitive impairment (MCI)

In each of the relevant aspects and embodiments of the invention, the exosomes are derived from Flk-1⁺ progenitor cells. In particular, non-limiting aspects, the Flk-1⁺ exosomes are derived from vascular endothelial progenitor cells or the exosomes are derived from neural stem cells. As reported herein, these exosomes specifically target neural cells in the brain

In each of the relevant aspects and embodiments of the invention, the therapeutically-effective amount of exosomes is a population of between 1×10⁶ and 1×10¹² exosomes, administered daily or once a week. In certain aspects, the therapeutically-effective amount of exosomes is a population of 5×10⁹ exosomes.

In each of the relevant aspects and embodiments of the invention, the “preventing”, “reducing” and “abrogating” is at least 50%, in comparison to a subject not undergoing one of the methods of the invention. Each of the “preventing”, “reducing” and “abrogating” may also be at least 50%, at least 60%, at least 70%, at least 80%, at least 90%, at least 95%, at least 99%, or 100%, in comparison to a subject not undergoing the one of the methods of the invention.

Exosomes are small, extracellular vesicles of 50-150 nm in diameter that mediate cell-to-cell communication. Because exosomes are noncytotoxic, have low immunogenicity, are easy to store, have a long lifespan, and high cargo loading capacity, they have been proposed to be nanometric vehicles for therapeutic agent and gene delivery [120]. The mechanism underlying exosome-specific targeting is still unclear. One potential mechanism involves membrane receptors on the exosomes, which bind cell surface proteins of the recipient cells [121].

In each of the relevant aspects and embodiments of the invention, the exosomes used in the methods of the invention are exosome isolated from Flk-1⁺ vascular endothelial progenitor cells and/or neural stem cells. The exosomes may be harvested from the cells by means well known in the art, such as those described in the Examples herein.

In each of the relevant aspects and embodiments of the invention, the “therapeutically-effective amount of exosomes” is an amount of exosomes sufficient to achieve the goal of the method, i.e. treating or preventing AD and AD-related dementia, delaying onset of symptoms of Alzheimer's disease, suppressing ASK1 activity in the brain of a subject, or inducing glucose metabolism in a neuron or neuronal cell. The therapeutically-effective amount of exosomes, when the exosomes are isolated from Flk-1⁺ vascular endothelial progenitor cells and/or neural stem cells, may also be defined as ranging from about 1×10⁶ to about 1×10¹² exosomes, from about 1×10⁷ to about 1×10¹¹ exosomes, or from about 1×10⁸ to about 1×10¹⁰ exosomes, or being about 1×10⁹ exosomes, 5×10⁹ exosomes, or 1×10¹⁰ exosomes.

Alternatively, the “therapeutically-effective amount of exosomes containing Trx1 polypeptide” may be defined based on the total amount of agent or therapeutic agent delivered to a neuron or neuronal cell.

The subjects on which the methods of the invention may be practiced are mammals, e.g. a human, a non-human primate, horse, cow, goat, sheep, a companion animal, such as a dog, cat or rodent, or other mammal.

In the methods of the present invention, the exosomes may be administered to the subject at a frequency that includes a single administration to achieve the methods of the invention. Alternatively, a suitable number of administrations includes 1, 2, 3, 4, 5, 6, 7, 8, 9 or 10 administrations of the exosomes. When administered more than once, the frequency of administered may be 4, 3, 2 or 1 time per day, every 2, 3, 4, 5, 6, 7, 8, 9, 10, 11, 12 or 13 days, once weekly, every two weeks, every three weeks, every four weeks, once a month, every two months, every three month, every four months, or less common, for example.

When administered to the subject, the exosomes may be formulated, for example, for oral, sublingual, intranasal, intraocular, rectal, transdermal, mucosal, pulmonary, topical (e.g., via a skin patch) or parenteral administration. The exosomes may be formulated in a suitable diluent, for example, phosphate buffered saline (PBS). Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

In each of the relevant aspects and embodiments of the invention, the delivery is targeted delivery, for example in which the exosomes target neurons or neuronal cells in the brain.

The exosomes may be administered to the subject via any means suitable in the art. The exosomes may be formulated in a suitable diluent, for example, phosphate buffered saline (PBS).

### III. Examples

Experiment 1—To determine whether inhibiting the ASK1-p38MAPK pathway prevents insulin resistance during AD pathogenesis and leads to alleviation of AD symptoms.

ASK1, a protein kinase, directly responds to oxidative stress [27,37]. The endogenous redox protein thioredoxin 1 (Trx1) binds to and thus prevents ASK1 activation [27,37]. Increased reactive oxygen species (ROS) and the activation of p38MAPK, a kinase downstream of ASK1, are involved in the pathogenesis of AD [66]. ASK1 is a member of the mitogen-activated protein-kinase kinase-kinase (MAPKKK) family that activates the p38MAPK and c-Jun N-terminal kinase 1/2 (JNK1/2) signaling pathways [67,68]. Amyloid-beta (Aβ) is a major factor leading to AD pathology and ROS-activated ASK1 mediates the apoptotic effect of Aβ in neurons [69]. These findings suggest that ASK1 may contribute to the pathogenesis of AD.

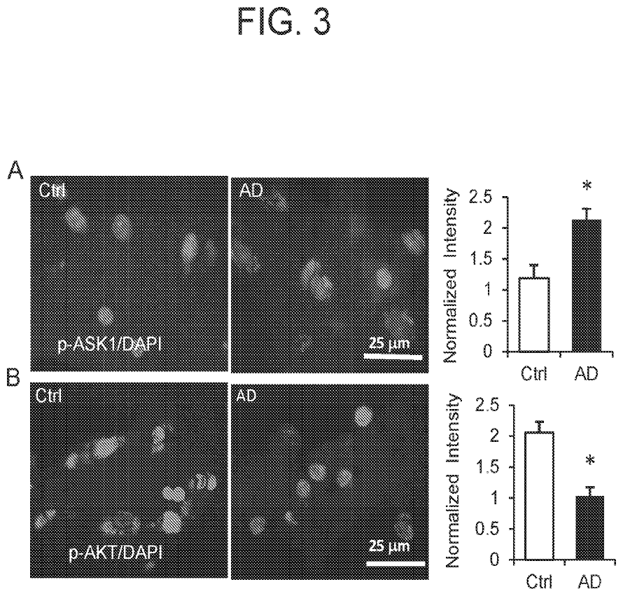

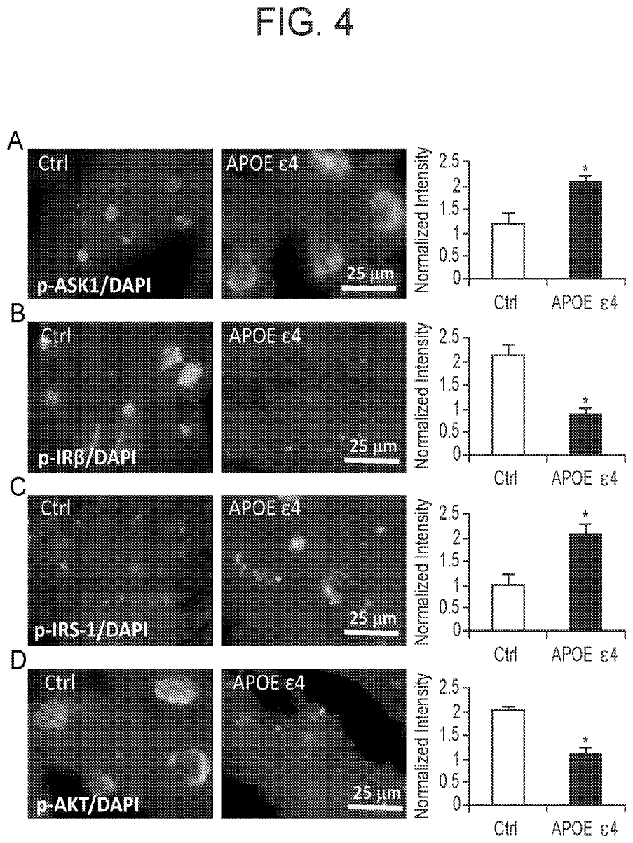

Indeed, a recent study has demonstrated the direct involvement of ASK1 in AD pathogenesis [1]. ASK1 deficiency improves cognitive function in a mouse model of AD, which was shown to be associated with the reduction of phosphorylated p38MAPK [1]. However, the mechanism underlying ASK1-induced AD pathology is still unknown. ASK1 was activated in postmortem AD frontal cortices and hippocampi with a cooccurrence of reduced IR signaling (FIG. 3). ASK1 activation and impaired IR signaling were also observed in the frontal cortices and hippocampi from non-AD individuals who were carriers of APOE ε4 (FIG. 4), which is the strongest genetic risk factor for AD [28]. Four cases of APOE ε4 brain tissues were obtained (APOE ε3/4 (3 cases) and APOE ε4/4(1 case), all males, ages from 43-54 and causes of death are heart diseases (2 cases), drug (1), and natural (1)), and four cases of APOE 3 control brain tissues (all males, ages from 39-52, and causes of death are heart disease (1), drug (2), and pontine hemorrhage (1)).

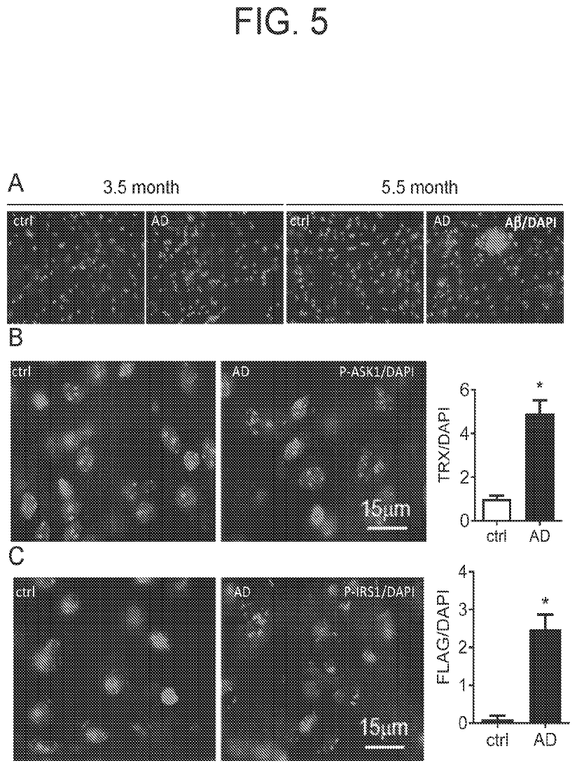

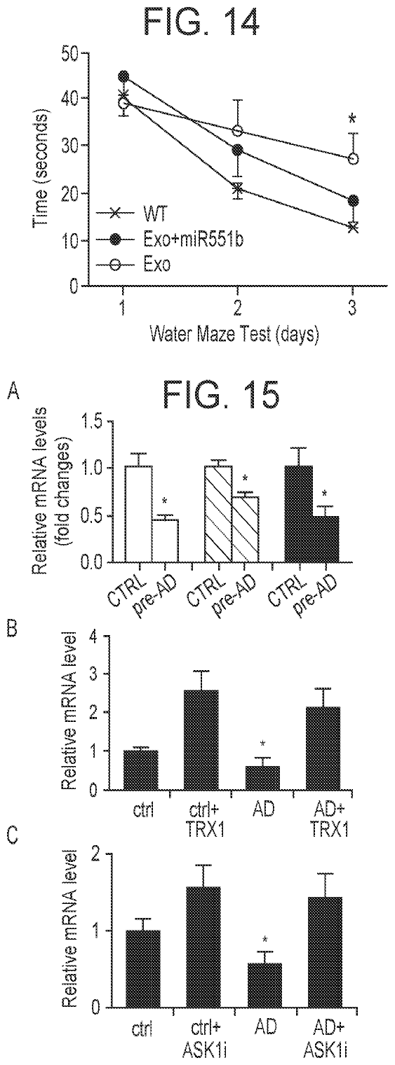

Furthermore, it was found that in the APP/PS1 mouse model of AD, Aβ deposits (FIG. 5A) and cognitive impairment occurred at 5.5 months of age (FIG. 2 and FIG. 14), whereas ASK1 activation and IR signaling impairment were present at 3.5 months of age (FIG. 5B, C); importantly, this latter finding preceded AD manifestation, which occurs at 5.5 months of age onwards. Therefore, it was hypothesized that ASK1 activation inhibits IR signaling, brain insulin resistance, glucose uptake and mitochondrial function, leading to the progression of AD pathology.

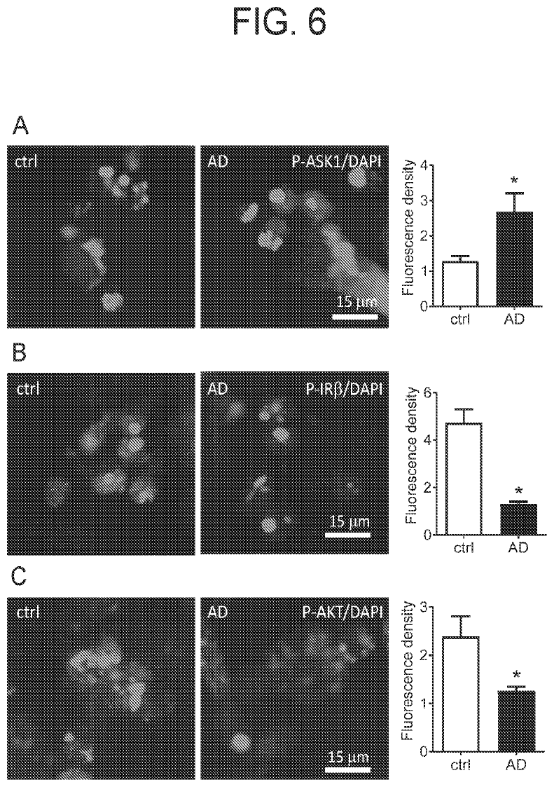

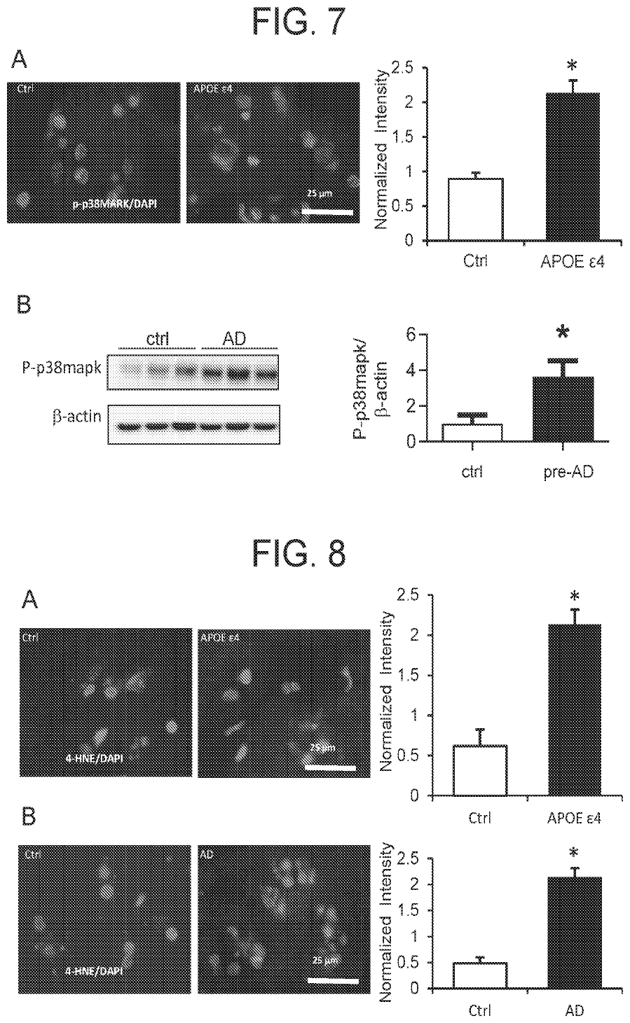

Phosphorylation at tyrosine residues of the insulin receptor substrates IRS-1 and IRS-2 is an early and important process of insulin signal transduction [70]. Impaired tyrosine phosphorylation and increased serine phosphorylation of IRS-1 are correlated with insulin resistance in vivo [71]. ASK1 overexpression inhibits IRS-1 activity by increasing its serine phosphorylation and decreasing insulin-stimulated tyrosine phosphorylation of IRS-1 [38], whereas dominant-negative ASK1 overexpression abrogates TNFα-inhibited IRS-1 activity [38]. It was found that IRβ phosphorylation at tyrosine 1146 (Tyr1146) and Akt phosphorylation were reduced, whereas serine phosphorylation of IRS-1 (p-Ser-IRS-1) and the phosphorylation of ASK1 and p38MAPK were increased in the postmortem tissue samples of the frontal cortices and hippocampi from non-AD individuals who were carriers of APOE ε4 (FIG. 4, 7A), AD-patient iPS cells (iPSCs) (FIG. 6), and 3.5-month-old APP/PS1 mice (FIG. 5, 7B). Activated IRS-1 upon tyrosine phosphorylation by IR leads to the activation of the PI3K-Akt pathway [72]. The activity of GSK-3β, a downstream effector of the PI3K/Akt pathway, can be inhibited by Akt-mediated phosphorylation at Ser9 [73]. Due to the downregulation of insulin signaling and the PI3K/Akt pathway, GSK-3β Ser9 (the inactive form of GSK-3β) was reduced in the above three conditions (data not included). These findings suggest that insulin resistance and the activation of the ASK1-p38MAPK pathway precede overt AD symptoms. Thus, it is proposed that blocking ASK1-p38MAPK will mitigate insulin resistance and ameliorate AD pathology during neural differentiation of AD iPSCs and the progression of AD pathogenesis in the employed AD mouse model.

Interestingly, iPSCs derived from skin fibroblasts of AD patients recapitulate the proposed process of AD pathogenesis in which the activation of the ASK1-p38MAPK pathway suppresses insulin signaling leading to AD manifestation (FIG. 6). To test whether ASK1 deficiency in iPSCs will prevent insulin signaling impairment and AD manifestation during neural differentiation, ASK1-knockout iPSC lines will be generated using CRISPR/Cas9. ASK1 gRNAs (5′-UUGCAAUCAAGAAGCUCAGC-3′) will be cloned into px459 v1 (Addgene plasmid number Plasmid #48139) containing 2A-PURO selection and will be electroporated (Amaxa, Lonza) into iPSCs. After electroporation, cells will be plated at a clonal density onto feeder-mouse embryonic fibroblasts (MEFs) and will be subjected to puromycin selection (2 μg/ml) after 36-48 h. Single colonies will be picked and tested for the loss of ASK1 by immunoblotting analysis, and clones will be confirmed by the sequencing of PCR products.

Healthy control human iPSCs and AD human iPSCs (detailed in Table 1) purchased from the Coriell Institute for Medical Research, Camden, NJ, will be cultured in 0.1% gelatin-coated plates with CF-1 MEF feeder cells in maintenance medium: knockout DMEM/F12, 10% knockout serum replacement, 1× glutamine, 1× NEAA, 1× β-mercaptoethanol, 10 ng/ml basic FGF, and 1× penicillin/streptomycin. Additional iPSC lines (3 AD and 3 non-AD) have been made from AD patient's skin fibroblasts purchased from Coriell. The proposed experiments will include healthy young females (30-45 years), healthy old (≥60 years) males and females, young and old familial AD females, old sporadic females and males. Thus, age and sex will be controlled in both familial and sporadic ADs.

TABLE 1
iPSCs derived from fibroblasts of AD and healthy individuals

| | | Affected | | |
|---|---|---|---|---|
| ID | Health Condition | Status | Sex | Age |
| AG25367 | FAMILIAL AD | YES | Female | 31 YR |
| AG25370 | FAMILIAL AD | YES | Female | 81 YR |
| AG11415 | SPORADIC AD | YES | Male | 55 YR |
| AG07376 | SPORADIC AD | YES | Male | 60 YR |
| GM24675 | SPORADIC AD | YES | Female | 60 YR |
| GM24666 | SPORADIC AD | YES | Male | 83 YR |
| GM25430 | APPARENTLY HEALTHY | NO | Male | 62 YR |
| GM00967 | APPARENTLY HEALTHY | NO | Male | 51 YR |
| GM23248 | APPARENTLY HEALTHY | NO | Male | 55 YR |
| GM23279 | APPARENTLY HEALTHY | NO | Female | 36 YR |
| GM23280 | APPARENTLY HEALTHY | NO | Female | 36 YR |
| GM23720 | APPARENTLY HEALTHY | NO | Female | 22 YR |

To eliminate the influence of high glucose [74-76] and to faithfully reflect in vivo conditions, iPSCs will be adapted to normal glucose (5 mM), as has been previously described [74,75]. Next, iPSCs will be initially transferred to a medium containing 11.1 mM glucose for eight passages and subsequently will be subjected to 8.3 mM glucose for an additional eight passages. The resultant iPSCs will then be transferred to medium with 5 mM glucose for 4 passages and will subsequently be used for experiments. The neural differentiation procedure that will be employed has been described previously [104,105]. Briefly, iPSC colonies will be dissociated by collagenase IV (Sigma) and washed three times in growth media. The colonies will be resuspended in growth media without bFGF in an uncoated bacterial petri dish to form embryonic bodies (EBs) for 7 days. Then, EBs will be plated on polyornithine/laminin-coated plates in DMEM/F12 supplemented with N2 and bFGF until rosette formation. The rosette will be manually removed and dissociated into single cells with TryPLE (Invitrogen), and it will then be cultured in DMEM/F12 supplemented with N2, B27-RA, and 20 ng/mL of bFGF on polyornithine/laminin-coated plates. For final neural differentiation, the medium will be changed to differentiation medium (DMEM with F12+Glutamax, 0.5× B27, 0.5× N2 (Invitrogen), penicillin/streptomycin, 20 ng/ml BDNF, 20 ng/ml GDNF (Peprotech), and 0.5 mM dibutyryl-cyclic AMP (Sigma)) until mature neuron and glial cell formation occurs.

To determine the impact of ASK1 deficiency on insulin signaling, iPSCs, iPSC-derived neural stem cells, and neurons will be subjected to measurements of both basal and insulin-stimulated activity of the IR→IRS-1→PI3K→Akt and the IR→IRS-1→RAS→ERK1/2 pathways. Specifically, 1 nM and 10 nM insulin will be used to treat cells, the concentrations of which represent physiological and supraphysiological insulin concentrations in the human brain [21]. Next, immunoblotting will be employed to measure the following: pTyr1146; pTyr1150 and pTyr1151 of IRβ; tyrosine and serine phosphorylation of IRS-1; the phosphorylation of Akt; GSK-3β; RAS; and ERK1/2. The interaction of IRβ with IRS-1, as well as IRS-1 with the p85 subunit of PI3K will be assessed by immunoprecipitation. The interaction of IRβ with IRS-1 and IRS-1 with the p85 subunit of the PI3K will be assessed in immunoprecipitation. Since insulin signaling is impaired in AD cells, cell survival and cell proliferation supported by the two IR-IRS-1 downstream pathways, the PI3K-Akt and RAS-ERK1/2 pathways, respectively, should be affected.

To investigate whether ASK1 deficiency will restore cell proliferation and neural lineage differentiation and to block apoptosis in AD-derived cells, the efficacy of iPSC differentiation into neurons and glial cells will be assessed. Cell proliferation will be measured by the Alamar Blue assay and 5-bromo-2′-deoxyuridine (BrdU) incorporation, followed by FACS analysis. Cell apoptosis will be determined by Annexin V staining and cleaved caspase 3 using immunoblotting. The abundance of neural stem cell (NSC) markers (Sox1 and Nestin), neuronal-specific markers (MAP2 and Tuj1), and a glial-cell marker (GFAP), will be determined using quantitative PCR. The numbers of NSCs, neurons and glial cells will be determined by immunostaining of specific markers and will be quantified via FACS analysis, as previously described [75,76]. Insulin signaling affects mitochondrial function and Akt, downstream of IR signaling, which phosphorylates and thus inactivates BAD [77], a pro-apoptotic member of the Bcl-2 family.

To examine whether ASK1 deficiency will restore mitochondrial dysfunction in AD cells, mitochondrial function indices will be analyzed. First, the number of defective mitochondria will be measured using electron microscopy (EM), as previously described [78]. Mitochondria that are considered defective will be those displaying increased size, ruptured outer membranes, and clear matrices due to cristolysis (the breakdown of internal membranous folds and disorganized cristae). Second, superoxide and hydrogen peroxide levels will be detected in total cellular lysates, cytosolic fractions and mitochondrial fractions. Third, the degree of mitochondrial depolarization will be evaluated. Mitochondria will be isolated using the Pierce Mitochondria Isolation Kit. The purity of the mitochondrial fraction will be assessed by immunoblotting using the mitochondrial marker prohibitin. Mitochondrial membrane potential (MMP) will be measured in isolated mitochondria using the fluorescent probe Rh-123. Fluorescent signals of the mitochondrial suspension will be analyzed by flow cytometry. Fourth, the levels of proapoptotic Bcl-2 members (Bad, Bid, Bax, and Bim) in the mitochondrial fraction, cytochrome C release to the cytoplasm, and caspase activation in total cellular lysates will be determined. Finally, mitochondrial respiration will be evaluated by measuring oxygen consumption using the Seahorse XF24 Analyzer, as previously described [79]. Sequential measurement of basal respiration (in the presence of substrate but no ADP), State 3 (+ADP), State 4ₒ (+oligomycin), and State 3ᵤ (+FCCP) will be performed on isolated mitochondria. Total cellular ATP production will be determined by the ATP Assay Kit (Abcam).

To determine the effect of ASK1 deficiency on glucose uptake and glucose transporters in AD cells, cells will be treated with the fluorescently labeled glucose analog 2-N-(7-nitro-benz-2-oxa-1,3-diazol-4-yl) amino)-2 deoxyglucose (2-NBDG; Invitrogen) for glucose uptake measurements, as previously described [80,81]. Cells will be incubated with or without 1 nM insulin for 15 min in the presence of 75 μM 2-NBDG for 30 min. Fluorescent intensity will be measured in a microplate reader. The membrane and cytosolic fractions of glucose transporters, mainly Glut1, Glut3 and Glut4, will be assessed by immunostaining and cellular fractionation, followed by immunoblotting.

To analyze the effect of ASK1 deficiency on the ontogeny of AD neuropathology during iPSC differentiation, Aβ and phosphorylated tau will be determined by immunostaining. ASK1 deficiency will be confirmed by Q-PCR analysis of ASK1 mRNA and immunoblotting of ASK1 protein. Additionally, p-p38MAPK and JNK1/2, two downstream effectors of ASK1 signal transduction, will be assessed.

To determine whether Ask1 gene deletion in the AD mouse model will ameliorate insulin signaling impairment and AD neuropathology, ASK1 knockout (KO) mice will be used [3,27,82]. ASK1 KO mice will be bred with the AD mouse line APP/PS1 with the C57/B6J background [83,84](purchased from the Jackson Laboratory, Stock No. 005864). The APP/PS1 AD mouse model comprises double-transgenic mice expressing a chimeric mouse/human amyloid precursor protein (Mo/HuAPP695swe) and a mutant human presenilin 1 (PS1-dE9) in central nervous system neurons[83,84]. Both mutations are associated with early-onset Alzheimer's disease. This AD mouse line is a useful model in the study of neuropathology, amyloid-plaque formation and aging in AD pathogenesis. To generate Wild-Type (WT), ASK1^{-/-}, APP/PS1, and APP/PS1/ASK1^{-/-} mice for experiments, ASK1^{+/-} (female) and APP/PS1/ASK1^{+/-} (male) mice will be bred. The activity of IR signaling will be analyzed by immunostaining and immunoblotting in isolated frontal cortices and hippocampi from 3.5-, 5.5- and 15-month-old mice. Cell proliferation will be determined by Ki67 and p-Histone H3 staining, and cell apoptosis will be evaluated by the TUNEL assay and cleaved caspase 3 staining. Mitochondrial function indices will be analyzed in isolated neurons. Aβ deposits will be detected using the 6E10 antibody (Biolegend, San Diego, CA). Astrogliosis will be visualized after staining with the rabbit monoclonal anti-GFAP antibody (Abcam).

To determine whether ASK1 deletion will restore glucose uptake and insulin sensitivity in the AD brain, positron emission tomography with 2-deoxy-2-[fluorine-18]fluoro-D-glucose integrated with computed tomography ([18F]FDG PET/CT) will be used. In vivo basal and insulin-stimulated [18F]FDG distributions in the mouse brain will be determined as has previously described [85]. Mice will be fasted overnight, [18F]FDG 11.1 MBq (300 Ci) and insulin (0 or 1.125 units/kg) in 200 l of saline will be injected via the tail vein under anesthesia. Dynamic imaging will be acquired for 30 min by the Siemens Inveon Small Animal PET/CT Imaging System. The data will be obtained by drawing regions of interest (ROIs) in the brain. The [18F]FDG biodistribution in brain regions will be analyzed at three time points: 10, 20, and 30 min. To detect insulin sensitivity in ex vivo, fresh frontal cortices and hippocampi will be isolated in oxygenated artificial cerebrospinal fluid buffer and then incubated with 1 or 10 nM insulin for 10 min. Tissues will be lysed for immunoblotting to assess IR signaling, as previously described [4].

To examine whether Ask1 gene deletion will alleviate cognitive impairment in AD mice, the Morris Water Maze (MWM) will be used for the assessment of spatial learning and memory at 5.5 and 15 months of age. The MWM test will be performed as previously described [88]. In this assessment, animals will learn to swim to a hidden platform under the water. Mice will be placed in the pool and will be allowed to explore it for 1 min. The testing room will be equipped with spatial cues for orientation. This training procedure will be performed 6 times a day for four consecutive days per animal. On day five, the platform will be removed from the pool to measure the time that the animals spend in the target quadrant until the platform is found by the animal. Additionally, during the training phase, the time spent finding the platform will also be measured for each animal. Learning performance will be measured as total latency, and the memory performance on the third and fourth days of training will also be determined. Sex as a biological variable: sex differences have been observed in the AD mouse model [89]. The data will be analyzed based on sex. Specifically, iPSCs from both sexes will be used, and data will be analyzed based on sex.

Whether inhibition of p38MAPK activation ameliorates insulin signaling impairment and AD development will be investigated. p38MAPK is the major downstream effector of ASK1 activation [1]. p38MAPK activation is activated in early-stage AD brains [57,90]. The inhibition of p38MAPK activation is considered a promising therapy in the treatment of AD, and various p38MAPK inhibitors tested in AD animal models show beneficial effects [2]. However, a causal role of p38MAPK activation in AD pathogenesis has not been demonstrated. It is hypothesized that the inhibition of p38MAPK activation will restore insulin signaling and alleviate AD pathogenesis. A unique model will be used, which is the p38AF mouse line (Jax Stock No: 012736). Heterozygous mice for the p38AF (p38AF^{+/-}) dominant-negative (DN) allele are viable and fertile, with two amino-acid substitutions (T180A and Y182F) at the activating phosphorylation sites of the p38MAPK locus [91,92]. However, homozygous p38AF is embryonically lethal. Importantly, p38AF^{+/-} mice exhibit specifically attenuated p38MAPK signaling without any reported effects on other MAPK pathways [91,92]. To generate WT, APP/PS1, p38AF^{+/-} and APP/PS1/p38AF^{+/-} mice for experiments, p38AF^{+/-} (female) and APP/PS1/p38AF^{+/-} (male) mice will be bred.

To determine whether p38MAPK inhibition will restore insulin signaling, prevent brain insulin resistance and alleviate AD pathology, indices of IR signaling, brain region glucose transporter and uptake, ex vivo insulin sensitivity, mitochondrial function, AD neuropathology and cognitive impairment will be analyzed.

To investigate whether p38MAPK inhibition will attenuate IR signaling impairment and AD neuropathology during AD-derived iPSC neural differentiation, iPSCs with stable DN p38MAPK expression will be established, as described for ASK1 KO iPSCs. Briefly, a DN p38MAPK vector will be electroporated into iPSCs, which will be subjected to puromycin selection. Single colonies will be tested for DN p38MAPK expression. The activity of IR signaling intermediates, glucose uptake and transporter expression, mitochondrial function indices, AD neuropathy indices, cell proliferation, apoptosis, and differentiation will be assessed.

Anticipated results, pitfalls, and alternatives: it is expected that Ask1 gene deletion will restore insulin signaling, insulin sensitivity, glucose uptake, glucose transporter expression and mitochondrial function in AD-derived iPSCs and in the hippocampi and frontal cortices of APP/PS1 mice. In the absence of ASK1, apoptosis, impaired cell proliferation, and neural differentiation in AD-derived iPSCs will be abrogated, and consequently, AD neuropathology, including Aβ and phosphorylated tau, will be significantly reduced in neurons derived from AD iPSCs. In the APP/PS1 mice, early insulin signaling restoration (3.5 months of age) will be translated into the amelioration of AD neuropathology and cognitive impairment at 5.5 and 15 months of age. Similarly, it is anticipated that the attenuation of p38MAPK activity in p38AF^{+/-} mice will prevent insulin resistance, glucose uptake and mitochondrial function, leading to the alleviation of AD neuropathology and cognitive impairment. Two complementary AD models will be used, human AD-derived iPSCs in neural differentiation and the AD mouse model APP/PS1, to faithfully reflect human AD pathogenesis. These two models will sufficiently serve the objective in revealing the effect of the ASK1-p38MAPK pathway on insulin signaling impairment and AD pathogenesis. The APP/PS1 mouse line has been widely used in AD research [93-97] and has the same genetic background, C57/B6J, as the genetically modified mice. Aβ deposits occur at 5.5 months, and behavioral performance decreases soon after in APP/PS1 mice. Additional AD mouse models, such as the 5×FAD model (MMRRC Stock No: 34840-JAX), will be used as an alternative approach if the two models do not meet the needs of the proposed studies in terms of elucidating the roles of kinase and insulin signaling. Based on the strong preliminary data, this scenario is unlikely. ASK1 KO mice do not show any adverse phenotypes [3,27,82]. One alternative approach is to test the effect of the ASK1 inhibitor selonsertib [56] on insulin signaling impairment in AD pathogenesis. The other downstream kinase of ASK1, JNK1/2, is also activated prior to overt AD neuropathology (data not shown). If the effect of p38MAPK inhibition is not prominent, whether the deletion of the Jnk1 gene or the Jnk2 gene restores insulin signaling and prevents AD pathology in human iPSCs and APP/PS1 mice will be examined. JNK1 KO and JNK2 KO mice from previous studies will be used [29,30].

Experiment 2—To investigate the effect of restoring thioredoxin 1 and its targeted exosomal delivery on brain ASK1 activation, insulin resistance and AD pathogenesis.

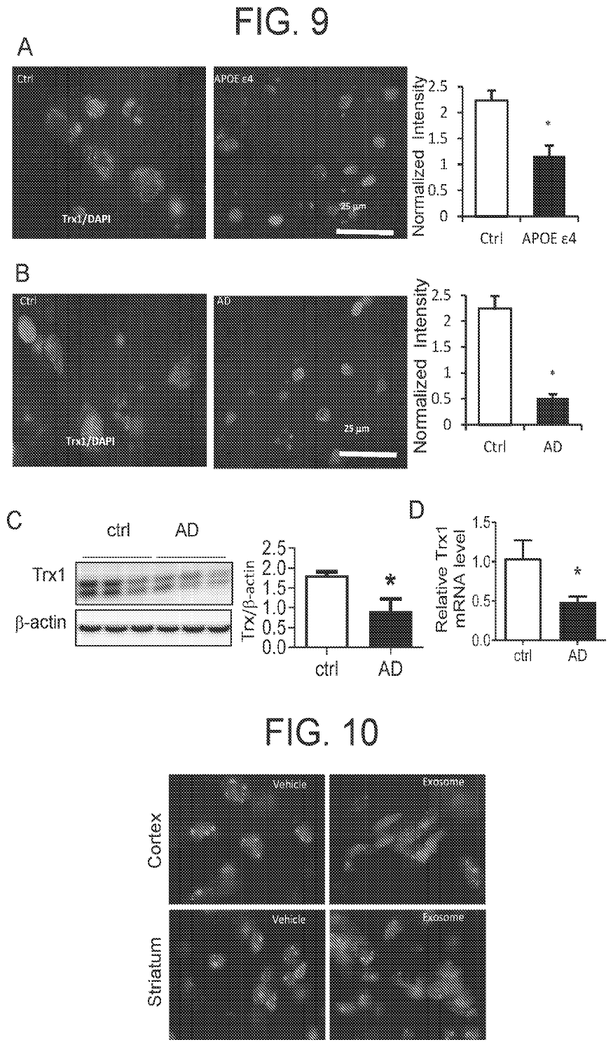

The redox protein thioredoxin 1 (Trx1) is the endogenous inhibitor of ASK1 [67]. In response to ROS, Trx1 is oxidized and dissociated from ASK1, leading to ASK1 autophosphorylation and activation [67]. It was observed that the oxidative stress marker 4-hydroxynonenal (HNE) was significantly increased in postmortem tissue samples of the frontal cortices and hippocampi from APOE ε4-carrying non-AD individuals (FIG. 8A) and from AD patients (FIG. 8B), suggesting that ROS build-up before AD manifestation may reduce Trx1 expression and activity by the oxidation of its cysteine residues, leading to ASK1 activation. It has been reported that Trx1 protein is significantly reduced in early-stage AD brains compared to that of age-matched control brains [98,99]. In contrast to the Trx1 reduction in AD brain regions, the levels of Trx1 are increased in AD cerebrospinal fluid, which may be due to Trx1 shedding from neurons [100]. In a targeted-replacement mouse model, human APOE ε4, the major genetic risk factor for AD, suppresses Trx1 expression in the hippocampus [101]. Human recombinant APOE ε4 represses Trx1 expression in human primary cortical neurons [101]. It was also found that Trx1 was significantly decreased in postmortem brain tissue samples from non-AD APOE ε4 carriers (FIG. 9A), from AD patients (FIG. 9B), and from 1.5-month-old APP/PS1 AD mice (FIG. 9C, D). AD iPSCs also demonstrated lower Trx1 expression than control subjects (data not included). These observations support an important role of Trx1 reduction in AD pathogenesis.

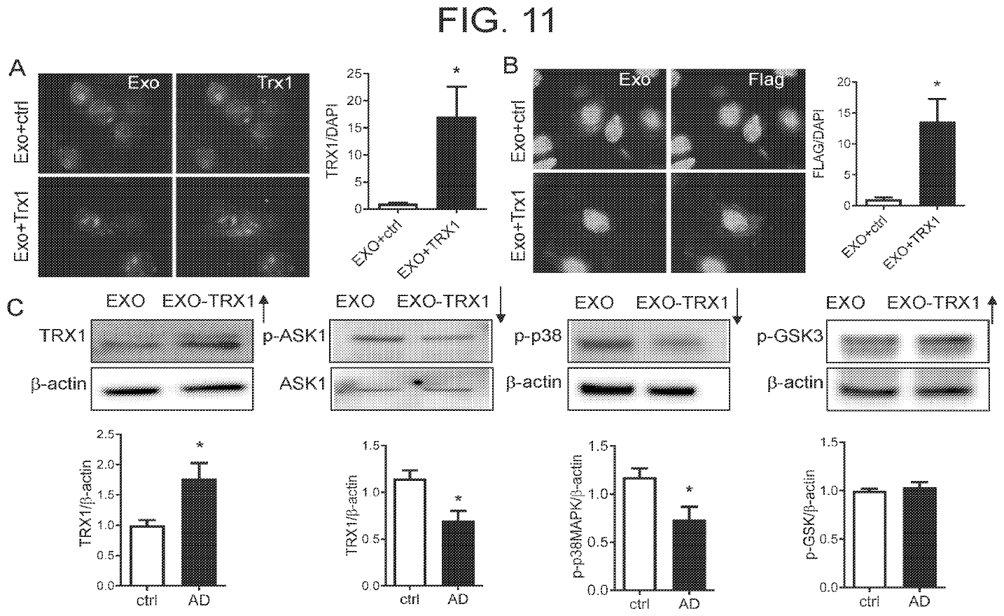

Indeed, studies have revealed that Trx1 overexpression inhibits Aβ neurotoxicity in vitro [99]. Trx1 overexpression also blocks APOE ε4 toxicity to neurons [101]. Since Trx1 downregulation occurred earlier than the manifestation of AD neuropathy (FIG. 9A, C, D), it was hypothesized that the restoration of Trx1 expression at an earlier stage before overt AD neuropathology will restore insulin signaling and alleviate AD pathogenesis. A Trx1 Tg mouse model specifically in neural tissues will be developed to test this hypothesis. It has been shown that a truncated Trx1 is expressed in neurons and can be packaged into exosomes produced by neurons [102]. An exosomal delivery approach will be designed for Trx1 to treat AD. It has been shown that exosomes from a certain type of cell specifically target another cell type, which uptakes these exosomes and uses exosomal cargos, including mRNAs and miRNAs, to improve cellular function [8,103]. Exosomes from Flk1 (VEGF receptor 2)-positive (Flk1⁺) vascular progenitors specifically target brain cells (FIG. 10). Trx1 mRNA-loaded Flk1⁺ exosomes delivered to mice via tail-vein injections were taken up by cells in the brain (FIG. 11A, B), leading to increased Trx1 protein expression in the brain (FIG. 11A, B, C) and ultimately resulting in the blockage of ASK1 activation (FIG. 11C) and the restoration of insulin signaling by increasing the level of GSK-3β Ser9 (the inactive form of GSK-3β) (FIG. 11C). Whether exosomal Trx1 mRNA delivery will restore IR signaling and thus ameliorate AD progression will be tested.

To determine whether Trx1 overexpression abolishes ASK1 activation, leading to the restoration of insulin signaling, the prevention of insulin resistance and the amelioration of AD pathogenesis, NSC-marker Nestin promoter-driven Trx1 Tg mice will be generated. The rat nestin promoter construct was provided by Dr. Weimin Zhong (Yale University) [104]. Under this nestin promoter, it has been consistently demonstrated that transgene expression occurs at embryonic day 8 (E8) onwards, and specifically in the neuroepithelium containing primarily NSCs [105]. Thus, transgene expression covers the entire spectrum of neural development and neural lineages. The Trx1 expression vector will be co-constructed with a nuclear sequence-tagged enhanced green fluorescent protein (EGFP) that is co-expressed with Trx1. The transgene will express two separate products, Trx1 and nuclear EGFP, which will be used for the monitoring of transgene expression. To generate WT, Trx1 Tg, APP/PS1, and APP/PS1/Trx1 mice for experiments, Trx1 Tg (female) and APP/PS1 (male) mice will be bred. The activation of ASK1 and its downstream kinases (p38MAPK and JNK1/2), as well as indices of IR signaling and mitochondrial function, glucose transport/uptake, ex vivo insulin sensitivity, cell proliferation and apoptosis, Aβ deposition and astrogliosis, will be assessed in isolated frontal cortices and hippocampi at 3.5, 5.5 and 15 months of age. Cognitive function evaluated by the MWM test will be performed at 5.5 and 15 months of age.

To test whether Trx1 overexpression will abolish the activation of the ASK1 pathway and, thus, inhibit insulin resistance and AD neuropathology during iPSC neural differentiation, iPSCs with stable Trx1 overexpression will be established as described for ASK1 KO iPSCs. Briefly, a Trx1 expression vector will be electroporated into iPSCs, which will be subjected to puromycin selection. Single colonies will be tested for Trx1 expression. The activity of ASK1-p38MAPK/JNK1/2, IR signaling intermediates, insulin sensitivity, glucose uptake, and glucose transporter expression, mitochondrial function indices, AD neuropathy indices, cell proliferation, apoptosis, and differentiation will be assessed in non-AD and AD iPSCs, with or without Trx1 overexpression.

To elucidate the inhibitory effect of Trx1-enriched exosomes on ASK1 activation, insulin resistance, and AD development, Trx1 mRNA-enriched exosomes will be injected through the tail veins of mice at 3 months of age as described in the preliminary study (FIG. 11). A Trx1 cDNA vector containing the T7 promoter in 5′ and hGH poly(A) signal in 3′ (Origen, Rockville, MD) will be used for Trx1 mRNA in vitro transcription using the mMESSAGE mMACHINE™ T7 Transcription Kit (Thermo Scientific). Trx1 mRNA will be purified by the Megaclear™ Kit (Thermo Scientific). The control mRNA will be the nonconserved Xenopus elongation factor 1α provided in the mMESSAGE mMACHINE™ T7 Transcription Kit. Exosomes will be isolated from culture media of mouse embryonic Flk1⁺ endothelial progenitors using Total Exosome Isolation Reagent (Thermo Scientific). The size and concentrations of exosomes will be measured in the NanoSight NS300 System (Malvern Panalytical). Two micrograms of Trx1 mRNA and control mRNA will be loaded into 10⁷ exosomes using the Exo-Fect™ Exosome Transfection Kit (System Biosciences). Total RNA will be isolated (exoRNEasy kit, Qiagen, Inc) from Trx1 and control mRNA-loaded exosomes. Comparative analysis of the presence of Trx mRNA in these two different exosomes will be performed by quantitative RT-PCR.

Specifically, 10⁷ exosomes/day/mouse for seven days will be injected into the following groups: (1) WT mice injected with the control mRNA-enriched EXOs; (2) WT mice injected with the Trx1 mRNA-enriched EXOs; (3) APP/PS1 AD mice injected with the control mRNA-enriched EXOs; and (4) APP/PS1 AD mice injected with the Trx1 mRNA-enriched EXOs. Since insulin signaling impairment occurs at 3.5 months of age, mice will be treated at three months of age to determine whether the suppression of early insulin resistance by Trx1 ameliorates later-occurring AD pathogenesis. One week after the last injections (3.5 months of age), the hippocampi and the frontal cortices of mouse brains will be analyzed for the activation of the ASK1 pathway, the ASK1-Trx1 complex, insulin sensitivity, glucose transporter/uptake, mitochondrial dysfunction, cell apoptosis, and proliferation. Two separate groups of EXO-treated mice will be analyzed at 5.5 and 15 months of age for cognitive function, Aβ deposition and astrogliosis in addition to the above cellular indices. Previous studies have demonstrated that injected EXOs are retained in the targeted tissues for 28 days [8,103]. Thus, the injected EXOs in this system will have long-lasting effects. EXOs are labeled by the green fluorescent dye Pkh67 (Sigma, Cat #Mini67), and Trx1 mRNA has an HA tag for the monitoring of Trx1 expression resulting from exosomal Trx1 mRNA. Injected EXOs will be detected in brain regions as performed in the preliminary data (FIG. 11).

Anticipated results, pitfalls, and alternatives: It is anticipated that Trx1 Tg overexpression in neural tissues from early embryonic development will block ASK1 and p38MAPK activation, brain insulin resistance, mitochondrial dysfunction, and neuron apoptosis and will restore glucose uptake and glucose transporter expression in the hippocampi and frontal cortices of AD mice at 3.5 months of age. These beneficial effects elicited by Trx1 Tg overexpression will translate into the prevention or significant amelioration of AD neuropathology at 5.5 and 15 months of age. Stable Trx1 overexpression will inhibit the activation of the ASK1-p38MAPK pathway, block apoptosis, and restore insulin sensitivity, glucose uptake and transporter expression, mitochondrial function and cell proliferation in AD iPSCs and their progeny NSCs. In iPSC-derived neurons, Trx1 overexpression prevents or significantly alleviates AD neuropathology and neuronal apoptosis. Since Trx1 is an endogenous inhibitor of ASK1 and is an antioxidant, it is expected that Trx1 overexpression either in Tg mice or in stable iPSC lines will terminate the adverse effect of the ASK1-initiating pathway on insulin signaling/sensitivity, leading to AD pathology. However, other endogenous antioxidant components, such as glutaredoxin (Grx), are also altered in postmortem AD brain tissues [100]. The glutathione (GSH)-Grx system is a powerful redox balance system and GSH is the most abundant small molecular thiol and is present at a high concentration in the brain [106]. If Trx1 overexpression has only marginal benefits in combating insulin resistance in AD, the hypothesis will be tested by overexpressing Grx or double-overexpressing Trx1 and Grx1. It is expected that tail-vein delivery of Trx1 mRNA-loaded exosomes will abrogate the activation of the ASK1-p38MAPK signaling and reverse insulin signaling impairment, mitochondrial dysfunction and neuronal apoptosis. Exosomal Trx1 delivery ultimately suppresses AD neuropathology and improves cognitive function in AD mice. Based on previous reports, the regimen of injecting 10⁹ exosomes/day for one week will be used, and using this approach, the preliminary study demonstrated efficacy in blocking ASK1 activation and brain insulin resistance in AD mice (FIG. 11). However, if it turns out that the efficacy of Trx1 mRNA-loaded exosomes is not robust, the treatment regime will be optimized by using different amounts of exosomes and different durations of treatment. A recent study shows that allogeneic exosomes do not induce immunoreactions [8,103]. If immunoreactions are observed upon allogeneic exosome treatment, immunosuppression will be used in the exosome-treatment study.

Experiment 3—To determine whether miR-551b reduction contributes to brain insulin resistance during AD pathogenesis.

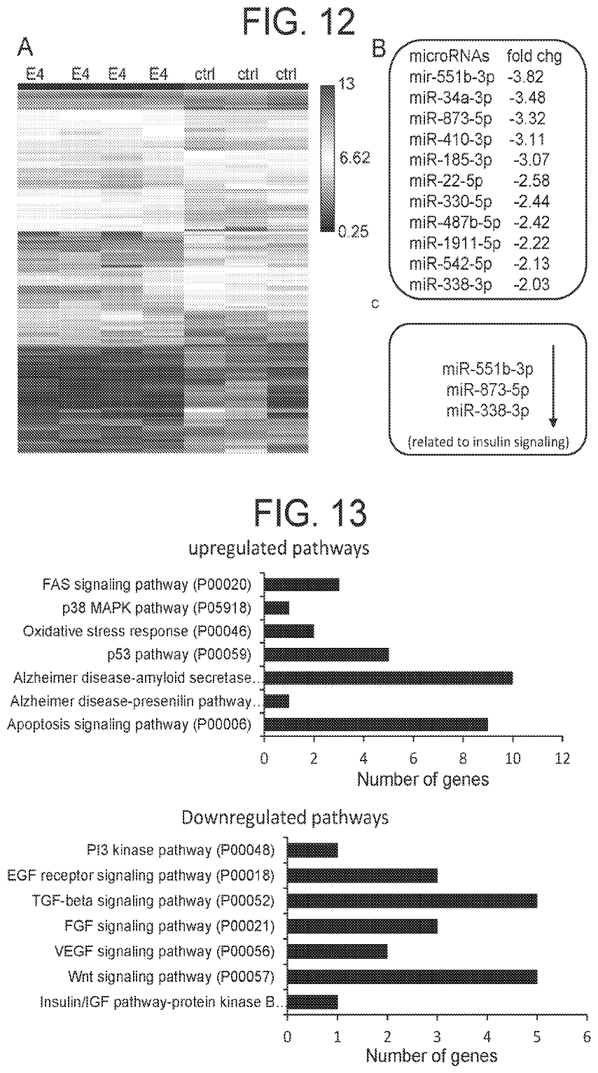

miRNAs are small (21-25 nucleotides) and noncoding endogenous RNAs that posttranscriptionally repress gene expression by degrading mRNA and/or inhibiting its translation [107]. It has been demonstrated that miRNAs play important roles in neural development and differentiation and that approximately 70% of all miRNAs are expressed in the brain. Extensive studies have shown that the expression of hundreds of miRNAs is altered in the AD brain [108,109]. However, the role of individual miRNAs in AD pathogenesis is still elusive. To reveal the miRNA alteration responsible for impaired insulin signaling, an miRNA profiling study was performed in the post-mortem hippocampi of non-AD individuals who were carriers of APOE ε4 (FIG. 12) and it was found that miRNAs were differentially regulated in APOE ε4 carriers compared to those in noncarriers (FIG. 12). Furthermore, in a transcriptome microarray study, it was found that oxidative stress, p38MAPK and the AD pathways were among the top upregulated pathways, whereas the PI3 kinase-Akt pathway and the insulin/IGF pathway were among the top downregulated pathways in non-AD APOE ε4 carriers (FIG. 13). Mouse homologs of the most downregulated miRNAs in the human APOE ε4 study were further verified in the hippocampi of 1-, 1.5- and 3.5-month-old APP/PS1 AD mice (FIG. 15A) and it was found that miR-551b, miR-383, and miR-872 were downregulated at the time of ASK1 activation and subsequent insulin signaling impairment (FIG. 12C). Among them, miR-551b is particularly interesting because it is among the few miRNAs that are consistently downregulated in studies of human AD postmortem samples [109]. Additionally, it has been shown that miR-551b deficiency leads to decreased Akt activity [110], which is a major readout of insulin receptor signaling, whereas miR-551b overexpression enhances insulin signaling by activating Akt [110]. Therefore, it was hypothesized that the decrease in miR-551b that precedes overt AD pathology is responsible for impaired insulin signaling/sensitivity and that the restoration of miR-551b expression prevents insulin resistance and ameliorates AD progression. Preliminary data support this hypothesis by demonstrating that the delivery of miR-551b-enriched exosomes to APP/PS1 AD mice significantly improved cognitive function in AD mice (FIG. 14). It has been shown that ASK1 activation modulates miRNA expression in microglia [111] and that the ASK1 downstream kinase p38MAPK also regulates miRNA expression in cancer cells [112]. It was demonstrated that the endogenous ASK1 inhibitor Trx1 abrogated the downregulation of miR-551b in human AD iPSCs (FIG. 15B) and that the ASK1 inhibitor selonsertib restored miR-551b expression in human AD iPSCs (FIG. 15C). Thus, it was hypothesize that the activation of the ASK1-p38MAPK signaling pathway leads to miR-551b downregulation.

To determine whether ASK1 and p38MAPK are essential for miR-551b downregulation, the miR-551b levels will be assessed in ASK1 KO and p38MAPK KO iPSCs, iPSC-derived neural stem cells and neurons. The miRNA genes are transcribed to long primary miRNAs (pri-miRNAs), which are processed by Drosha to form precursor miRNAs (pre-miRNAs) [113]. A precursor miRNA produces a mature miRNA, a guide strand for gene regulation, and a passenger strand, which is degraded and does not play a role in gene regulation [114,115]. Pri-miR-551b, pre-miR-551b, and mature miR-551b will be measured via Q-PCR. The transcription factors downstream of the p38MAPK signaling are ATF2, MEF2, DDIT3 and the tumor suppressor p53 [116]. The binding activities of these transcription factors to the miR-551b promoter will be examined using chromatin immunoprecipitation (ChIP). ChIP will be carried out by using antibodies against these transcription factors to pull-down DNA-transcription factor complexes. To examine whether Ask1 deletion or p38MAPK inhibition by p38AF restores miR-551b expression in the AD mouse model, miR-551b abundance and transcription factor binding to the miR-551b promoter will be assessed in frontal cortices and hippocampi from WT, APP/PS1, ASK1^{-/-}, APP/PS1/ASK1^{-/-}, p38AF^{+/-}, and APP/PS1/p38AF^{+/-} mice.

To test whether miR-551b overexpression restores insulin sensitivity and thus alleviates AD pathogenesis, nestin-promoter-driven miR-551b Tg mice will be generated, as described in Example 2 for the Trx1 Tg mice. To generate WT, miR-551b Tg, APP/PS1, and APP/PS1/miR-551 Tg mice for experiments, miR-551b Tg (female) and APP/PS1 (male) mice will be bred. Indices of insulin receptor signaling, ex vivo insulin sensitivity, mitochondrial function, glucose transport/uptake, cell proliferation and apoptosis, as well as Aβ deposition and astrogliosis, will be determined in isolated frontal cortices and hippocampi at 3.5, 5.5 and 15 months of age. Cognitive function evaluated by the MWM test will be performed at 5.5, and 15 months of age.

To determine whether miR-551b overexpression will prevent insulin resistance and ameliorate AD neuropathology during iPSCs neural differentiation, iPSCs with stable expression of the miR-551b mimic or a control miRNA mimic will be generated through lentiviral infections, which will be purchased from Dharmacon. IR signaling intermediates, insulin sensitivity, glucose uptake, glucose transporter expression, mitochondrial function indices, AD neuropathy indices, and cell proliferation, apoptosis, and differentiation will be assessed in non-AD and AD iPSCs, with or without miR-551b overexpression. To identify miR-551b target genes, a biotin-labeled miRNA pulldown assay will be performed, as it has been previously described [105]. iPSCs will be transfected with a biotin-labeled miR-551b (Dharmacon, Lafayette, CO, USA), and cell lysates will be mixed with streptavidin-coupled Dynabeads (Invitrogen). Bead-bound RNA duplexes (miR-551b-mRNAs) will be isolated for RNAseq. miR-551b target genes will be annotated with the Ingenuity Pathway Analysis (IPA) platform. IPA will be queried with these targets, aiming to map and generate putative biological processes/functions, networks and pathways based on the manually curated knowledge database of molecular interactions extracted from the public literature. The gene network will be generated using both direct and indirect relationships/connectivity as previously described [117]. These networks will be ranked by scores that will measure the probability that the genes will be included in the network by chance alone.

To investigate the efficacy of miR-551b-enriched exosomes (EXOs) on inhibiting insulin resistance and the amelioration of AD development, miR-551b-enriched EXOs will be injected through the tail veins of mice at three months of age. Two micrograms of miR-551b or a control miRNA will be loaded into 10⁹ Flk1⁺ endothelial progenitor EXOs, as described in Example 2 for Trx1-mRNA exosome loading. A total of 10⁹ exosomes/day/mouse for 7 days will be injected into the following groups: 1) WT mice injected with the control miRNA-enriched EXOs; 2) WT mice injected with the miR-551b-enriched EXOs; 3) APP/PS1 AD mice injected with the control miRNA-enriched EXOs; and 4) APP/PS1 AD mice injected with the miR-551b-enriched EXOs. One week after the last injections (3.5 months of age), the hippocampus and the frontal cortex of mouse brains will be analyzed for insulin sensitivity, glucose transporter/uptake, and cell apoptosis and proliferation. Two separate groups of EXO-treated mice will be analyzed at 5.5 and 15 months of age for cognitive function, A p deposition and astrogliosis in addition to the above cellular indices.

Anticipated results, pitfalls, and alternatives: It is expected that either deleting the Ask1 gene or inhibiting p38MAPK in p38AF mice will restore miR-551b expression in the AD mouse brain. Likewise, CRISPR-Cas knockout of the Ask1 gene or the p38Mapk gene will prevent miR-551b downregulation in human AD iPSCs and their neural progenies. Enhanced transcription factor binding to the mir551b promoter in AD iPSCs and the AD mouse brain will be abolished upon deleting Ask1 or inhibiting p38MAPK. One potential problem is the functional readout of the transcription factor binding to the mir351b promoter. The ChIP assay will reveal the binding activities of ATF2, MEF2, DDIT3, and p53 with the mir551b promoter and may not entail functional interaction. Alternatively, luciferase reporter experiments will be conducted, as previously described [105], to confirm the inhibitory effect of these transcription factors on the mir551b promoter activity. It is anticipated that miR-551b Tg expression will restore insulin signaling/sensitivity, glucose uptake and transporter expression and cellular function in 3.5-month-old AD mice, leading to the amelioration of AD neuropathology and the improvement of cognitive function at 5.5 and 15 months of age. Similarly, expression of the miR-551b mimic in AD iPSCs restores insulin sensitivity and cellular function, leading to the alleviation of AD neuropathology in neurons derived from AD iPSCs. The miR-551b pulldown assay will reveal target genes of this miRNA in AD iPSCs, and the top pathways regulated by miR-551b target genes will be insulin sensitivity, mitochondrial function and AD pathology. It is expected that the therapeutic arms of tail-vein delivery of miR-551b-loaded exosomes will reverse the impairment of insulin signaling and cellular function in the hippocampus and frontal cortex of AD mice at 3.5 months of age, leading to prevention of AD neuropathology and cognitive impairment at 5.5 and 15 months of age. If miR-551b overexpression does not affect insulin resistance and AD pathology, the role of other downregulated miRNAs discovered in the microarray study (FIG. 12) will be investigated. These miRNAs include miR-383 and miR-873, and it has been reported that these miRNAs can suppress insulin resistance [118] and positively regulate Akt activity downstream of IR signaling [119]. Alternatively, the combination of two or three miRNAs may be used to enhance the therapeutic efficacy of miRNAs against brain insulin resistance and AD pathology in the study.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
[1] Hasegawa Y, Toyama K, Uekawa K, Ichijo H, Kim-Mitsuyama S: Role of ASK1/p38 Cascade in a Mouse Model of Alzheimer's Disease and Brain Aging. J Alzheimers Dis 2018, 61:259-63.
[2] Kheiri G, Dolatshahi M, Rahmani F, Rezaei N: Role of p38/MAPKs in Alzheimer's disease: implications for amyloid beta toxicity targeted therapy. Rev Neurosci 2018, 30:9-30.
[3] Wu Y, Wang F, Fu M, Wang C, Quon M J, Yang P: Cellular Stress, Excessive Apoptosis, and the Effect of Metformin in a Mouse Model of Type 2 Diabetic Embryopathy. Diabetes 2015, 64:2526-36.
[4] Arnold S E, Lucki I, Brookshire B R, Carlson G C, Browne C A, Kazi H, Bang S, Choi B R, Chen Y, McMullen M F, Kim S F: High fat diet produces brain insulin resistance, synaptodendritic abnormalities and altered behavior in mice. Neurobiol Dis 2014, 67:79-87.
[5] Wakabayashi T, Yamaguchi K, Matsui K, Sano T, Kubota T, Hashimoto T, Mano A, Yamada K, Matsuo Y, Kubota N, Kadowaki T, Iwatsubo T: Differential effects of diet- and genetically-induced brain insulin resistance on amyloid pathology in a mouse model of Alzheimer's disease. Mol Neurodegener 2019, 14:15.
[6] Barzilai N, Ferrucci L: Insulin resistance and aging: a cause or a protective response? J Gerontol A Biol Sci Med Sci 2012, 67:1329-31.
[7] Liao Y, Du X, Li J, Lonnerdal B: Human milk exosomes and their microRNAs survive digestion in vitro and are taken up by human intestinal cells. Mol Nutr Food Res 2017, 61.
[8] Saha P, Sharma S, Korutla L, Datla S R, Shoja-Taheri F, Mishra R, Bigham G E, Sarkar M, Morales D, Bittle G, Gunasekaran M, Ambastha C, Arfat M Y, Li D, Habertheuer A, Hu R, Platt M O, Yang P, Davis M E, Vallabhajosyula P, Kaushal S: Circulating exosomes derived from transplanted progenitor cells aid the functional recovery of ischemic myocardium. Sci Transl Med 2019, 11.
[9] Association As: Alzheimer's Facts and Figures Report https://wwwalzorg/media/HomeOffice/Facts%20and%20Figures/facts-and-figurespdf 2018.
[10] Armstrong R A: The pathogenesis of Alzheimer's disease: a reevaluation of the “amyloid cascade hypothesis”. Int J Alzheimers Dis 2011, 2011:630865.
[11] Neth B J, Craft S: Insulin Resistance and Alzheimer's Disease: Bioenergetic Linkages. Front Aging Neurosci 2017, 9:345.
[12] Sherrington R, Rogaev E I, Liang Y, Rogaeva E A, Levesque G, Ikeda M, Chi H, Lin C, Li G, Holman K, Tsuda T, Mar L, Foncin J F, Bruni A C, Montesi M P, Sorbi S, Rainero I, Pinessi L, Nee L, Chumakov I, Pollen D, Brookes A, Sanseau P, Polinsky R J, Wasco W, Da Silva H A, Haines J L, Perkicak-Vance M A, Tanzi R E, Roses A D, Fraser P E, Rommens J M, St George-Hyslop P H: Cloning of a gene bearing missense mutations in early-onset familial Alzheimer's disease. Nature 1995, 375:754-60.
[13] Citron M, Westaway D, Xia W, Carlson G, Diehl T, Levesque G, Johnson-Wood K, Lee M, Seubert P, Davis A, Kholodenko D, Motter R, Sherrington R, Perry B, Yao H, Strome R, Lieberburg I, Rommens J, Kim S, Schenk D, Fraser P, St George Hyslop P, Selkoe D J: Mutant presenilins of Alzheimer's disease increase production of 42-residue amyloid beta-protein in both transfected cells and transgenic mice. Nat Med 1997, 3:67-72.
[14] Facchini F S, Hua N, Abbasi F, Reaven G M: Insulin resistance as a predictor of age-related diseases. J Clin Endocrinol Metab 2001, 86:3574-8.
[15] Reiman E M, Caselli R J, Yun L S, Chen K, Bandy D, Minoshima S, Thibodeau S N, Osborne D: Preclinical evidence of Alzheimer's disease in persons homozygous for the epsilon 4 allele for apolipoprotein E. N Engl J Med 1996, 334:752-8.
[16] Small G W, Ercoli L M, Silverman D H, Huang S C, Komo S, Bookheimer S Y, Lavretsky H, Miller K, Siddarth P, Rasgon N L, Mazziotta J C, Saxena S, Wu H M, Mega M S, Cummings J L, Saunders A M, Pericak-Vance M A, Roses A D, Barrio J R, Phelps M E: Cerebral metabolic and cognitive decline in persons at genetic risk for Alzheimer's disease. Proc Natl Acad Sci USA 2000, 97:6037-42.
[17] Sperling R A, Aisen P S, Beckett L A, Bennett D A, Craft S, Fagan A M, Iwatsubo T, Jack C R, Jr., Kaye J, Montine T J, Park D C, Reiman E M, Rowe C C, Siemers E, Stern Y, Yaffe K, Carrillo M C, Thies B, Morrison-Bogorad M, Wagster M V, Phelps C H: Toward defining the preclinical stages of Alzheimer's disease: recommendations from the National Institute on Aging-Alzheimer's Association workgroups on diagnostic guidelines for Alzheimer's disease. Alzheimers Dement 2011, 7:280-92.
[18] Hoyer S: Is sporadic Alzheimer disease the brain type of non-insulin dependent diabetes mellitus? A challenging hypothesis. J Neural Transm (Vienna) 1998, 105:415-22.
[19] Steen E, Terry B M, Rivera E J, Cannon J L, Neely T R, Tavares R, Xu X J, Wands J R, de la Monte S M: Impaired insulin and insulin-like growth factor expression and signaling mechanisms in Alzheimer's disease—is this type 3 diabetes? J Alzheimers Dis 2005, 7:63-80.
[20] Rivera E J, Goldin A, Fulmer N, Tavares R, Wands J R, de la Monte S M: Insulin and insulin-like growth factor expression and function deteriorate with progression of Alzheimer's disease: link to brain reductions in acetylcholine. J Alzheimers Dis 2005, 8:247-68.
[21] Talbot K, Wang H Y, Kazi H, Han L Y, Bakshi K P, Stucky A, Fuino R L, Kawaguchi K R, Samoyedny A J, Wilson R S, Arvanitakis Z, Schneider J A, Wolf B A, Bennett D A, Trojanowski J Q, Arnold S E: Demonstrated brain insulin resistance in Alzheimer's disease patients is associated with IGF-1 resistance, IRS-1 dysregulation, and cognitive decline. J Clin Invest 2012, 122:1316-38.
[22] Reger M A, Watson G S, Frey W H, 2nd, Baker L D, Cholerton B, Keeling M L, Belongia D A, Fishel M A, Plymate S R, Schellenberg G D, Cherrier M M, Craft S: Effects of intranasal insulin on cognition in memory-impaired older adults: modulation by APOE genotype. Neurobiol Aging 2006, 27:451-8.
[23] Craft S, Baker L D, Montine T J, Minoshima S, Watson G S, Claxton A, Arbuckle M, Callaghan M, Tsai E, Plymate S R, Green P S, Leverenz J, Cross D, Gerton B: Intranasal insulin therapy for Alzheimer disease and amnestic mild cognitive impairment: a pilot clinical trial. Arch Neurol 2012, 69:29-38.
[24] Pratico D, Uryu K, Leight S, Trojanoswki J Q, Lee V M: Increased lipid peroxidation precedes amyloid plaque formation in an animal model of Alzheimer amyloidosis. J Neurosci 2001, 21:4183-7.
[25] Feng Z, Qin C, Chang Y, Zhang J T: Early melatonin supplementation alleviates oxidative stress in a transgenic mouse model of Alzheimer's disease. Free Radic Biol Med 2006, 40:101-9.
[26] Evans J L, Maddux B A, Goldfine I D: The molecular basis for oxidative stress-induced insulin resistance. Antioxid Redox Signal 2005, 7:1040-52.
[27] Yang P, Li X, Xu C, Eckert R L, Reece E A, Zielke H R, Wang F: Maternal hyperglycemia activates an ASK1-FoxO3a-caspase 8 pathway that leads to embryonic neural tube defects. Sci Signal 2013, 6:ra74.
[28] Liu C C, Liu C C, Kanekiyo T, Xu H, Bu G: Apolipoprotein E and Alzheimer disease: risk, mechanisms and therapy. Nat Rev Neurol 2013, 9:106-18.
[29] Li X, Weng H, Xu C, Reece E A, Yang P: Oxidative stress-induced JNK1/2 activation triggers proapoptotic signaling and apoptosis that leads to diabetic embryopathy. Diabetes 2012, 61:2084-92.
[30] Li X, Xu C, Yang P: c-Jun NH2-terminal kinase 1/2 and endoplasmic reticulum stress as interdependent and reciprocal causation in diabetic embryopathy. Diabetes 2013, 62:599-608.
[31] Gu H, Yu J, Dong D, Zhou Q, Wang J Y, Fang S, Yang P: High Glucose-Repressed CITED2 Expression Through miR-200b Triggers the Unfolded Protein Response and Endoplasmic Reticulum Stress. Diabetes 2016, 65:149-63.
[32] Markesbery W R, Carney J M: Oxidative alterations in Alzheimer's disease. Brain Pathol 1999, 9:133-46.
[33] Markesbery W R: Oxidative stress hypothesis in Alzheimer's disease. Free Radic Biol Med 1997, 23:134-47.
[34] Subbarao K V, Richardson J S, Ang L C: Autopsy samples of Alzheimer's cortex show increased peroxidation in vitro. J Neurochem 1990, 55:342-5.
[35] Lovell M A, Ehmann W D, Butler S M, Markesbery W R: Elevated thiobarbituric acid-reactive substances and antioxidant enzyme activity in the brain in Alzheimer's disease. Neurology 1995, 45:1594-601.
[36] Markesbery W R, Lovell M A: Four-hydroxynonenal, a product of lipid peroxidation, is increased in the brain in Alzheimer's disease. Neurobiol Aging 1998, 19:33-6.
[37] Ichijo H, Nishida E, Irie K, ten Dijke P, Saitoh M, Moriguchi T, Takagi M, Matsumoto K, Miyazono K, Gotoh Y: Induction of apoptosis by ASK1, a mammalian MAPKKK that activates SAPK/JNK and p38 signaling pathways. Science 1997, 275:90-4.
[38] Imoto K, Kukidome D, Nishikawa T, Matsuhisa T, Sonoda K, Fujisawa K, Yano M, Motoshima H, Taguchi T, Tsuruzoe K, Matsumura T, Ichijo H, Araki E: Impact of mitochondrial reactive oxygen species and apoptosis signal-regulating kinase 1 on insulin signaling. Diabetes 2006, 55:1197-204.
[39] Shen Y H, Zhang L, Gan Y, Wang X, Wang J, LeMaire S A, Coselli J S, Wang X L: Up-regulation of PTEN (phosphatase and tensin homolog deleted on chromosome ten) mediates p38 MAPK stress signal-induced inhibition of insulin signaling. A cross-talk between stress signaling and insulin signaling in resistin-treated human endothelial cells. J Biol Chem 2006, 281:7727-36.
[40] Moloney A M, Griffin R J, Timmons S, O'Connor R, Ravid R, O'Neill C: Defects in IGF-1 receptor, insulin receptor and IRS-1/2 in Alzheimer's disease indicate possible resistance to IGF-1 and insulin signalling. Neurobiol Aging 2010, 31:224-43.
[41] Pei J J, Khatoon S, An W L, Nordlinder M, Tanaka T, Braak H, Tsujio I, Takeda M, Alafuzoff I, Winblad B, Cowburn R F, Grundke-Iqbal I, Iqbal K: Role of protein kinase B in Alzheimer's neurofibrillary pathology. Acta Neuropathol 2003, 105:381-92.
[42] Rickle A, Bogdanovic N, Volkman I, Winblad B, Ravid R, Cowburn R F: Akt activity in Alzheimer's disease and other neurodegenerative disorders. Neuroreport 2004, 15:955-9.
[43] Griffin R J, Moloney A, Kelliher M, Johnston J A, Ravid R, Dockery P, O'Connor R, O'Neill C: Activation of Akt/PKB, increased phosphorylation of Akt substrates and loss and altered distribution of Akt and PTEN are features of Alzheimer's disease pathology. J Neurochem 2005, 93:105-17.
[44] Avila J, Wandosell F, Hernandez F: Role of glycogen synthase kinase-3 in Alzheimer's disease pathogenesis and glycogen synthase kinase-3 inhibitors. Expert Rev Neurother 2010, 10:703-10.
[45] Hooper C, Killick R, Lovestone S: The GSK3 hypothesis of Alzheimer's disease. J Neurochem 2008, 104:1433-9.
[46] Bomfim T R, Forny-Germano L, Sathler L B, Brito-Moreira J, Houzel J C, Decker H, Silverman M A, Kazi H, Melo H M, McClean P L, Holscher C, Arnold S E, Talbot K, Klein W L, Munoz D P, Ferreira S T, De Felice F G: An anti-diabetes agent protects the mouse brain from defective insulin signaling caused by Alzheimer's disease-associated Abeta oligomers. J Clin Invest 2012, 122:1339-53.
[47] Yarchoan M, Toledo J B, Lee E B, Arvanitakis Z, Kazi H, Han L Y, Louneva N, Lee V M, Kim S F, Trojanowski J Q, Arnold S E: Abnormal serine phosphorylation of insulin receptor substrate 1 is associated with tau pathology in Alzheimer's disease and tauopathies. Acta Neuropathol 2014, 128:679-89.
[48] Alzheimer's A: 2016 Alzheimer's disease facts and figures. Alzheimers Dement 2016, 12:459-509.
[49] Miya Shaik M, Tamargo I A, Abubakar M B, Kamal M A, Greig N H, Gan S H: The Role of microRNAs in Alzheimer's Disease and Their Therapeutic Potentials. Genes (Basel) 2018, 9.
[50] Luo X, Yan R: Inhibition of BACE1 for therapeutic use in Alzheimer's disease. Int J Clin Exp Pathol 2010, 3:618-28.
[51] Golde T E, Koo E H, Felsenstein K M, Osborne B A, Miele L: gamma-Secretase inhibitors and modulators. Biochim Biophys Acta 2013, 1828:2898-907.
[52] Gandy S, Sano M: Alzheimer disease: Solanezumab-prospects for meaningful interventions in AD? Nat Rev Neurol 2015, 11:669-70.
[53] Ehret M J, Chamberlin K W: Current Practices in the Treatment of Alzheimer Disease: Where is the Evidence After the Phase III Trials? Clin Ther 2015, 37:1604-16.
[54] Reger M A, Watson G S, Green P S, Baker L D, Cholerton B, Fishel M A, Plymate S R, Cherrier M M, Schellenberg G D, Frey W H, 2nd, Craft S: Intranasal insulin administration dose-dependently modulates verbal memory and plasma amyloid-beta in memory-impaired older adults. J Alzheimers Dis 2008, 13:323-31.
[55] Li Y, Li L, Holscher C: Incretin-based therapy for type 2 diabetes mellitus is promising for treating neurodegenerative diseases. Rev Neurosci 2016, 27:689-711.
[56] Loomba R, Lawitz E, Mantry P S, Jayakumar S, Caldwell S H, Arnold H, Diehl A M, Djedjos C S, Han L, Myers R P, Subramanian G M, McHutchison J G, Goodman Z D, Afdhal N H, Charlton M R, Investigators G-U-: The ASK1 inhibitor selonsertib in patients with nonalcoholic steatohepatitis: A randomized, phase 2 trial. Hepatology 2017.
[57] Hensley K, Floyd R A, Zheng N Y, Nael R, Robinson K A, Nguyen X, Pye Q N, Stewart C A, Geddes J, Markesbery W R, Patel E, Johnson G V, Bing G: p38 kinase is activated in the Alzheimer's disease brain. J Neurochem 1999, 72:2053-8.
[58] Alam J, Blackburn K, Patrick D: Neflamapimod: Clinical Phase 2b-Ready Oral Small Molecule Inhibitor of p38alpha to Reverse Synaptic Dysfunction in Early Alzheimer's Disease. J Prev Alzheimers Dis 2017, 4:273-8.
[59] Scheltens P, Prins N, Lammertsma A, Yaqub M, Gouw A, Wink A M, Chu H M, van Berckel B N M, Alam J: An exploratory clinical study of p38alpha kinase inhibition in Alzheimer's disease. Ann Clin Transl Neurol 2018, 5:464-73.
[60] Rajendran L, Honsho M, Zahn T R, Keller P, Geiger K D, Verkade P, Simons K: Alzheimer's disease beta-amyloid peptides are released in association with exosomes. Proc Natl Acad Sci USA 2006, 103:11172-7.
[61] Fiandaca M S, Kapogiannis D, Mapstone M, Boxer A, Eitan E, Schwartz J B, Abner E L, Petersen R C, Federoff H J, Miller B L, Goetzl E J: Identification of preclinical Alzheimer's disease by a profile of pathogenic proteins in neurally derived blood exosomes: A case-control study. Alzheimers Dement 2015, 11:600-7 el.
[62] Yuyama K, Sun H, Usuki S, Sakai S, Hanamatsu H, Mioka T, Kimura N, Okada M, Tahara H, Furukawa J, Fujitani N, Shinohara Y, Igarashi Y: A potential function for neuronal exosomes: sequestering intracerebral amyloid-beta peptide. FEBS Lett 2015, 589:84-8.
[63] Xin H, Li Y, Chopp M: Exosomes/miRNAs as mediating cell-based therapy of stroke. Front Cell Neurosci 2014, 8:377.
[64] Xin H, Li Y, Cui Y, Yang J J, Zhang Z G, Chopp M: Systemic administration of exosomes released from mesenchymal stromal cells promote functional recovery and neurovascular plasticity after stroke in rats. J Cereb Blood Flow Metab 2013, 33:1711-5.
[65] Yuyama K, Sun H, Sakai S, Mitsutake S, Okada M, Tahara H, Furukawa J, Fujitani N, Shinohara Y, Igarashi Y: Decreased amyloid-beta pathologies by intracerebral loading of glycosphingolipid-enriched exosomes in Alzheimer model mice. J Biol Chem 2014, 289:24488-98.
[66] Munoz L, Ammit A J: Targeting p38 MAPK pathway for the treatment of Alzheimer's disease. Neuropharmacology 2010, 58:561-8.
[67] Saitoh M, Nishitoh H, Fujii M, Takeda K, Tobiume K, Sawada Y, Kawabata M, Miyazono K, Ichijo H: Mammalian thioredoxin is a direct inhibitor of apoptosis signal-regulating kinase (ASK) 1. EMBO J 1998, 17:2596-606.
[68] Tobiume K, Matsuzawa A, Takahashi T, Nishitoh H, Morita K, Takeda K, Minowa O, Miyazono K, Noda T, Ichijo H: ASK1 is required for sustained activations of JNK/p38 MAP kinases and apoptosis. EMBO Rep 2001, 2:222-8.
[69] Kadowaki H, Nishitoh H, Urano F, Sadamitsu C, Matsuzawa A, Takeda K, Masutani H, Yodoi J, Urano Y, Nagano T, Ichijo H: Amyloid beta induces neuronal cell death through ROS-mediated ASK1 activation. Cell Death Differ 2005, 12:19-24.
[70] Saltiel A R, Kahn C R: Insulin signalling and the regulation of glucose and lipid metabolism. Nature 2001, 414:799-806.
[71] Hotamisligil G S, Peraldi P, Budavari A, Ellis R, White M F, Spiegelman B M: IRS-1-mediated inhibition of insulin receptor tyrosine kinase activity in TNF-alpha- and obesity-induced insulin resistance. Science 1996, 271:665-8.
[72] Boucher J, Kleinridders A, Kahn C R: Insulin receptor signaling in normal and insulin-resistant states. Cold Spring Harb Perspect Biol 2014, 6.
[73] Baki L, Shioi J, Wen P, Shao Z, Schwarzman A, Gama-Sosa M, Neve R, Robakis N K: PS1 activates PI3K thus inhibiting GSK-3 activity and tau overphosphorylation: effects of FAD mutations. EMBO J 2004, 23:2586-96.
[74] Yang P, Chen X, Kaushal S, Reece E A, Yang P: High glucose suppresses embryonic stem cell differentiation into cardiomyocytes: High glucose inhibits E S cell cardiogenesis. Stem Cell Res Ther 2016, 7:187.
[75] Yang P, Shen W B, Reece E A, Chen X, Yang P: High glucose suppresses embryonic stem cell differentiation into neural lineage cells. Biochem Biophys Res Commun 2016, 472:306-12.
[76] Chen X, Shen W B, Yang P, Dong D, Sun W, Yang P: High Glucose Inhibits Neural Stem Cell Differentiation Through Oxidative Stress and Endoplasmic Reticulum Stress. Stem Cells Dev 2018, 27:745-55.
[77] Datta S R, Dudek H, Tao X, Masters S, Fu H, Gotoh Y, Greenberg M E: Akt phosphorylation of BAD couples survival signals to the cell-intrinsic death machinery. Cell 1997, 91:231-41.
[78] Yang P, Xu C, Reece E A, Chen X, Zhong J, Zhan M, Stumpo D J, Blackshear P J, Yang P: Tip60- and sirtuin 2-regulated MARCKS acetylation and phosphorylation are required for diabetic embryopathy. Nat Commun 2019, 10:282.
[79] Rogers G W, Brand M D, Petrosyan S, Ashok D, Elorza A A, Ferrick D A, Murphy A N: High throughput microplate respiratory measurements using minimal quantities of isolated mitochondria. PloS one 2011, 6:e21746.
[80] Palmer C S, Ostrowski M, Gouillou M, Tsai L, Yu D, Zhou J, Henstridge D C, Maisa A, Hearps A C, Lewin S R, Landay A, Jaworowski A, McCune J M, Crowe S M: Increased glucose metabolic activity is associated with CD4+ T-cell activation and depletion during chronic HIV infection. AIDS 2014, 28:297-309.
[81] Palmer C S, Anzinger J J, Butterfield T R, McCune J M, Crowe S M: A Simple Flow Cytometric Method to Measure Glucose Uptake and Glucose Transporter Expression for Monocyte Subpopulations in Whole Blood. J Vis Exp 2016.
[82] Wang F, Wu Y, Quon M J, Li X, Yang P: ASK1 mediates the teratogenicity of diabetes in the developing heart by inducing E R stress and inhibiting critical factors essential for cardiac development. Am J Physiol Endocrinol Metab 2015, 309:E487-99.
[83] Jankowsky J L, Fadale D J, Anderson J, Xu G M, Gonzales V, Jenkins N A, Copeland N G, Lee M K, Younkin L H, Wagner S L, Younkin S G, Borchelt D R: Mutant presenilins specifically elevate the levels of the 42 residue beta-amyloid peptide in vivo: evidence for augmentation of a 42-specific gamma secretase. Hum Mol Genet 2004, 13:159-70.
[84] Jankowsky J L, Slunt H H, Ratovitski T, Jenkins N A, Copeland N G, Borchelt D R: Co-expression of multiple transgenes in mouse CNS: a comparison of strategies. Biomol Eng 2001, 17:157-65.
[85] Sun X J, Kim S P, Zhang D, Sun H, Cao Q, Lu X, Ying Z, Li L, Henry R R, Ciaraldi T P, Taylor S I, Quon M J: Deletion of interleukin 1 receptor-associated kinase 1 (Irak1) improves glucose tolerance primarily by increasing insulin sensitivity in skeletal muscle. J Biol Chem 2017, 292:12339-50.
[86] Cao Q, Hersl J, La H, Smith M, Jenkins J, Goloubeva O, Dilsizian V, Tkaczuk K, Chen W, Jones L: A pilot study of FDG PET/C T detects a link between brown adipose tissue and breast cancer. BMC Cancer 2014, 14:126.
[87] Cao Q, Lu M, Huebner T, Dilsizian V, Chen W: 18F-FDG PET/CT in a rare malignant extraskeletal osteosarcoma. Clin Nucl Med 2013, 38:e367-9.
[88] Clark S M, Vaughn C N, Soroka J A, Li X, Tonelli L H: Neonatal adoptive transfer of lymphocytes rescues social behaviour during adolescence in immune-deficient mice. Eur J Neurosci 2018, 47:968-78.
[89] Ordonez-Gutierrez L, Fernandez-Perez I, Herrera J L, Anton M, Benito-Cuesta I, Wandosell F: AbetaPP/PS1 Transgenic Mice Show Sex Differences in the Cerebellum Associated with Aging. J Alzheimers Dis 2016, 54:645-56.
[90] Sun A, Liu M, Nguyen X V, Bing G: P38 MAP kinase is activated at early stages in Alzheimer's disease brain. Exp Neurol 2003, 183:394-405.
[91] Shreeram S, Hee W K, Demidov O N, Kek C, Yamaguchi H, Fornace A J, Jr., Anderson C W, Appella E, Bulavin D V: Regulation of ATM/p53-dependent suppression of myc-induced lymphomas by Wip1 phosphatase. J Exp Med 2006, 203:2793-9.
[92] Wong E S, Le Guezennec X, Demidov O N, Marshall N T, Wang S T, Krishnamurthy J, Sharpless N E, Dunn N R, Bulavin D V: p38MAPK controls expression of multiple cell cycle inhibitors and islet proliferation with advancing age. Dev Cell 2009, 17:142-9.
[93] Roy D S, Arons A, Mitchell T I, Pignatelli M, Ryan T J, Tonegawa S: Memory retrieval by activating engram cells in mouse models of early Alzheimer's disease. Nature 2016, 531:508-12.
[94] Kim H Y, Kim H V, Jo S, Lee C J, Choi S Y, Kim D J, Kim Y: EPPS rescues hippocampus-dependent cognitive deficits in APP/PS1 mice by disaggregation of amyloid-beta oligomers and plaques. Nat Commun 2015, 6:8997.
[95] Jo S, Yarishkin O, Hwang Y J, Chun Y E, Park M, Woo D H, Bae J Y, Kim T, Lee J, Chun H, Park H J, Lee D Y, Hong J, Kim H Y, Oh S J, Park S J, Lee H, Yoon B E, Kim Y, Jeong Y, Shim I, Bae Y C, Cho J, Kowall N W, Ryu H, Hwang E, Kim D, Lee C J: GABA from reactive astrocytes impairs memory in mouse models of Alzheimer's disease. Nat Med 2014, 20:886-96.
[96] Furman J L, Sama D M, Gant J C, Beckett T L, Murphy M P, Bachstetter A D, Van Eldik L J, Norris C M: Targeting astrocytes ameliorates neurologic changes in a mouse model of Alzheimer's disease. J Neurosci 2012, 32:16129-40.
[97] Joshi G, Chi Y, Huang Z, Wang Y: Abeta-induced Golgi fragmentation in Alzheimer's disease enhances Abeta production. Proc Natl Acad Sci USA 2014, 111:E1230-9.
[98] Lovell M A, Xie C, Gabbita S P, Markesbery W R: Decreased thioredoxin and increased thioredoxin reductase levels in Alzheimer's disease brain. Free Radic Biol Med 2000, 28:418-27.
[99] Akterin S, Cowburn R F, Miranda-Vizuete A, Jimenez A, Bogdanovic N, Winblad B, Cedazo-Minguez A: Involvement of glutaredoxin-1 and thioredoxin-1 in beta-amyloid toxicity and Alzheimer's disease. Cell Death Differ 2006, 13:1454-65.
[100] Arodin L, Lamparter H, Karlsson H, Nennesmo I, Bjornstedt M, Schroder J, Fernandes A P: Alteration of thioredoxin and glutaredoxin in the progression of Alzheimer's disease. J Alzheimers Dis 2014, 39:787-97.
[101] Persson T, Lattanzio F, Calvo-Garrido J, Rimondini R, Rubio-Rodrigo M, Sundstrom E, Maioli S, Sandebring-Matton A, Cedazo-Minguez A: Apolipoprotein E4 Elicits Lysosomal Cathepsin D Release, Decreased Thioredoxin-1 Levels, and Apoptosis. J Alzheimers Dis 2017, 56:601-17.
[102] Gil-Bea F, Akterin S, Persson T, Mateos L, Sandebring A, Avila-Carino J, Gutierrez-Rodriguez A, Sundstrom E, Holmgren A, Winblad B, Cedazo-Minguez A: Thioredoxin-80 is a product of alpha-secretase cleavage that inhibits amyloid-beta aggregation and is decreased in Alzheimer's disease brain. EMBO Mol Med 2012, 4:1097-111.
[103] Sharma S, Mishra R, Bigham G E, Wehman B, Khan M M, Xu H, Saha P, Goo Y A, Datla S R, Chen L, Tulapurkar M E, Taylor B S, Yang P, Karathanasis S K, Goodlett D R, Kaushal S: A Deep Proteome Analysis Identifies the Complete Secretome as the Functional Unit of Human Cardiac Progenitor Cells. Circ Res 2017, 120:816-34.
[104] Petersen P H, Zou K, Hwang J K, Jan Y N, Zhong W: Progenitor cell maintenance requires numb and numblike during mouse neurogenesis. Nature 2002, 419:929-34.
[105] Wang F, Xu C, Reece E A, Li X, Wu Y, Harman C, Yu J, Dong D, Wang C, Yang P, Zhong J, Yang P: Protein kinase C-alpha suppresses autophagy and induces neural tube defects via miR-129-2 in diabetic pregnancy. Nat Commun 2017, 8:15182.
[106] Ren X, Zou L, Zhang X, Branco V, Wang J, Carvalho C, Holmgren A, Lu J: Redox Signaling Mediated by Thioredoxin and Glutathione Systems in the Central Nervous System. Antioxid Redox Signal 2017, 27:989-1010.
[107] Bushati N, Cohen S M: microRNA functions. Annu Rev Cell Dev Biol 2007, 23:175-205.
[108] Cogswell J P, Ward J, Taylor I A, Waters M, Shi Y, Cannon B, Kelnar K, Kemppainen J, Brown D, Chen C, Prinjha R K, Richardson J C, Saunders A M, Roses A D, Richards C A: Identification of miRNA changes in Alzheimer's disease brain and CSF yields putative biomarkers and insights into disease pathways. J Alzheimers Dis 2008, 14:27-41.
[109] Moradifard S, Hoseinbeyki M, Ganji S M, Minuchehr Z: Analysis of microRNA and Gene Expression Profiles in Alzheimer's Disease: A Meta-Analysis Approach. Sci Rep 2018, 8:4767.
[110] Xu X, Wells A, Padilla M T, Kato K, Kim K C, Lin Y: A signaling pathway consisting of miR-551b, catalase and MUC1 contributes to acquired apoptosis resistance and chemoresistance. Carcinogenesis 2014, 35:2457-66.
[111] Song J, Lee J E: ASK1 modulates the expression of microRNA Let7A in microglia under high glucose in vitro condition. Front Cell Neurosci 2015, 9:198.
[112] Antoon J W, Nitzchke A M, Martin E C, Rhodes L V, Nam S, Wadsworth S, Salvo V A, Elliott S, Collins-Burow B, Nephew K P, Burow M E: Inhibition of p38 mitogen-activated protein kinase alters microRNA expression and reverses epithelial-to-mesenchymal transition. Int J Oncol 2013, 42:1139-50.
[113] Dykxhoorn D M: MicroRNAs and metastasis: little RNAs go a long way. Cancer Res 2010, 70:6401-6.
[114] Thomson D W, Bracken C P, Goodall G J: Experimental strategies for microRNA target identification. Nucleic Acids Res 2011, 39:6845-53.
[115] Gregory R I, Chendrimada T P, Cooch N, Shiekhattar R: Human RISC couples microRNA biogenesis and posttranscriptional gene silencing. Cell 2005, 123:631-40.
[116] Yang S H, Sharrocks A D, Whitmarsh A J: Transcriptional regulation by the MAP kinase signaling cascades. Gene 2003, 320:3-21.
[117] Dong D, Zhang Y, Reece E A, Wang L, Harman C R, Yang P: microRNA expression profiling and functional annotation analysis of their targets modulated by oxidative stress during embryonic heart development in diabetic mice. Reprod Toxicol 2016, 65:365-74.
[118] Dou L, Wang S, Sun L, Huang X, Zhang Y, Shen T, Guo J, Man Y, Tang W, Li J: Mir-338-3p Mediates Tnf-A-Induced Hepatic Insulin Resistance by Targeting PP4r1 to Regulate PP4 Expression. Cell Physiol Biochem 2017, 41:2419-31.
[119] Han G, Zhang L, Ni X, Chen Z, Pan X, Zhu Q, Li S, Wu J, Huang X, Wang X: MicroRNA-873 Promotes Cell Proliferation, Migration, and Invasion by Directly Targeting TSLC1 in Hepatocellular Carcinoma. Cell Physiol Biochem 2018, 46:2261-70.
[120] Srivastava, A. et al. Exploitation of Exosomes as Nanocarriers for Gene-, Chemo-, and Immune-Therapy of Cancer. J Biomed Nanotechnol 12, 1159-1173, doi:10.1166/jbn.2016.2205 (2016).
[121] Rana, S., Yue, S., Stadel, D. & Zoller, M. Toward tailored exosomes: the exosomal tetraspanin web contributes to target cell selection. Int J Biochem Cell Biol 44, 1574-1584, doi:10.1016/j.biocel.2012.06.018 (2012).

1. A method of treating and/or preventing Alzheimer's disease (AD) and AD-related dementia, comprising administering a therapeutically-effective amount of exosomes to a subject in need thereof, wherein the exosomes contain a therapeutic agent for AD and/or AD-related dementia.

2. A method of delaying onset of symptoms of AD and AD-related dementia, comprising administering a therapeutically-effective amount of exosomes to a subject in need thereof, wherein the exosomes contain a therapeutic agent for AD and/or AD-related dementia.

3. The method of claim 1, wherein the therapeutic agent is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitor, p38MARK CRISPR/Cas9 and gRNA, p38MARK siRNA, and p38MAPK inhibitor.

4. The method of claim 1, wherein the therapeutic agent is Trx1 mRNA, Trx1 protein, Trx1 core peptide, or miR-551b.

5. The method of claim 1, wherein the subject carries the APOE ε4 risk factor gene.

6. The method of claim 5, wherein the subject also has mild cognitive impairment (MCI).

7. The method of claim 1, wherein the exosomes are derived from Flk-1⁺ progenitor cells.

8. The method of claim 7, wherein the Flk-1⁺ exosomes are derived from vascular endothelial progenitor cells or the exosomes are derived from neural stem cells.

9. The method of claim 1, wherein the exosomes are administered to the subject via an intravenous injection or a skin patch.

10. A method of suppressing ASK1 activity in the brain of a subject, comprising administering a therapeutically-effective amount of exosomes to a subject in need thereof, wherein the exosomes contain a therapeutic agent that inhibits ASK1 gene transcription, inhibits ASK1 mRNA translation, or inhibits ASK1 protein activity.

11. The method of claim 10, wherein the therapeutic agent is one or more of thioredoxin-1 (Trx1) mRNA, Trx1 protein, Trx1 core peptide, miR-551b, miR-383, miR-873, apoptosis signal-regulating kinase 1 (ASK1) CRISPR/Cas9 and gRNA, ASK1 siRNA, ASK1 inhibitor, p38MAPK CRISPR/Cas9 and gRNA, p38MARK siRNA, and p38MAPK inhibitor.

12. The method of claim 10, wherein the therapeutic agent is Trx1 mRNA, Trx1 protein, Trx1 core peptide or miR-551b.

13. The method of claim 10, wherein the subject has AD or AD-related dementia, or the subject is at risk of developing AD.

14. The method of claim 13, wherein the subject carries the APOE ε4 risk factor gene.

15. The method of claim 14, wherein the subject also has mild cognitive impairment (MCI).

16. The method of claim 10, wherein the exosomes are derived from Flk-1⁺ progenitor cells.

17. The method of claim 16, wherein the Flk-1⁺ exosomes are derived from vascular endothelial progenitor cells or the exosomes are derived from neural stem cells.

18. The method of claim 10, wherein the exosomes are administered to the subject via an intravenous injection or a skin patch.

19-36. (canceled)
